SEQUENCE LISTING (2 sequences)

SEQ ID NO: 1 - protein, 493 aa, Artificial Sequence
  Made in Lab - synthesized anti-BMCA CAR
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1485 bp, Artificial Sequence
  Made in Lab - synthesized anti-BMCA CAR
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

(19) United States
(12) Patent Application Publication
(10) Pub. No.: US 2022/0195060 A1
(43) Pub. Date: Jun. 23, 2022

(54) MANUFACTURING ANTI-BCMA CAR T CELLS

(71) Applicant: 2seventy bio, Inc., Cambridge, MA (US)

(72) Inventors: Kevin FRIEDMAN, Melrose, MA (US); Eric Scott ALONZO, Melrose, MA (US)

(73) Assignee: 2seventy bio, Inc., Cambridge, MA (US)

(21) Appl. No.: 17/601,383

(22) PCT Filed: Apr. 2, 2020
(86) PCT No.: PCT/US2020/026300

§ 371 Date: Oct. 4, 2021

Related U.S. Application Data
(60) Provisional application No. 62/944,485, filed on Dec. 6, 2019.
(60) Provisional application No. 62/830,004, filed on Apr. 5, 2019.

(51) Int. Cl. C07K 16/28; C12N 5/0783; C12N 15/86; A61P 35/00; A61K 35/17; C07K 14/725

(52) U.S. Cl. CPC C07K 16/2878; C12N 5/0636; C12N 15/86; A61P 35/00; A61K 35/17; C07K 14/7051; A61K 2039/505; C12N 2506/11; C12N 2501/2302; C12N 2510/00; C07K 2319/30; C07K 2319/33; C12N 2740/16043

(57) ABSTRACT

The invention provides improved anti-BCMA CAR T cell compositions and methods for manufacturing anti-BCMA CAR T cell therapies. More particularly, the invention relates to improved methods of for manufacturing anti-BCMA CAR T cells that result in more potent, persistence, and efficacious adoptive T cell immunotherapies. In certain embodiments, the cells were manufactured from a subject that has a multiple myeloma or a lymphoma.

### CROSS REFERENCE TO RELATED APPLICATIONS

This application claims the benefit under 35 U.S.C. § 119(e) of U.S. Provisional Application No. 62/944,485, filed Dec. 6, 2019, and 62/830,004, filed Apr. 5, 2019, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_118_02WO_ST25.txt. The text file is 7 KB, was created on Mar. 27, 2020, and is being submitted electronically via EFS-Web, concurrent with the filing of the specification.

### BACKGROUND

### Technical Field

The present invention relates to improved anti-BCMA CAR T cell compositions and methods for manufacturing anti-BCMA CAR T cells. More particularly, the invention relates to improved methods of for manufacturing anti-BCMA CAR T cells that result in more potent, persistence, and efficacious adoptive T cell immunotherapies.

### Description of the Related Art

Adoptive immunotherapy is the transfer of T lymphocytes to a subject to provide therapy for a disease. Adoptive immunotherapy has yet unrealized potential for treating a wide variety of diseases including cancer, infectious disease, autoimmune disease, inflammatory disease, and immunodeficiency. However, most, if not all adoptive immunotherapy strategies require T cell activation and expansion steps to generate a clinically effective, therapeutic dose of T cells. Current technologies for generating therapeutic doses of T cells, including engineered T cells, remain limited by cumbersome T cell manufacturing processes. For example, T cell expansion often requires labor intensive and expensive cloning, and/or multiple rounds of activation/expansion to achieve therapeutically relevant T cell numbers. In addition, existing T cell activation/expansion methods are normally coupled with substantial T cell differentiation and usually result in short-lived effects, including short-lived survival and a lack of persistence and lack of in vivo expansion of the transferred T cells. More recent manufacturing methods have resulted in more potent and persistent T cells, but these cells are still prone to exhaustion and loss of effector immune cell function.

There is still an unmet need for improvements in T cell manufacturing and more potent and persistent T cell therapies.

### BRIEF SUMMARY

The invention generally provides adoptive T cell immunotherapies with improved potency and persistence and methods of making the same.

In various embodiments, a cGMP manufactured population of anti-B cell maturation antigen (BCMA) chimeric antigen receptor (CAR) T cells is provided that comprises at least 10% CD27⁺ anti-BCMA CAR T cells.

In particular embodiments, the population comprises at least 15% CD27⁺ anti-BCMA CAR T cells.

In certain embodiments, the population comprises at least 20% CD27⁺ anti-BCMA CAR T cells.

In some embodiments, the population comprises at least 25% CD27⁺ anti-BCMA CAR T cells.

In further embodiments, the population comprises at least 30% CD27⁺ anti-BCMA CAR T cells.

In particular embodiments, the CD27⁺ anti-BCMA CAR T cells are LEF1⁺ and/or TCF1⁺ anti-BCMA CAR T cells.

In additional embodiments, the CD27⁺ anti-BCMA CAR T cells are LEF1⁺ and TCF1⁺ anti-BCMA CAR T cells.In various embodiments, a cGMP manufactured population of anti- BCMA CAR T cells comprises at least 10% LEF1⁺ and/or CCR7⁺ and TCF1⁺ anti-BCMA CAR T cells.

In some embodiments, the population comprises at least 15% LEF1⁺ and/or CCR7⁺ and TCF1⁺ anti-BCMA CAR T cells.

In particular embodiments, the population comprises at least 20% LEF1⁺ and/or CCR7⁺ and TCF1⁺ anti-BCMA CAR T cells.

In some embodiments, the population comprises at least 25% LEF1⁺ and/or CCR7⁺ and TCF1⁺ anti-BCMA CAR T cells.

In further embodiments, the population comprises at least 30% LEF1⁺ and/or CCR7⁺ and TCF1⁺ anti-BCMA CAR T cells.

In additional embodiments, the LEF1⁺ and/or CCR7⁺ and/or TCF1⁺ anti-BCMA CAR T cells are CD27⁺ anti-BCMA CAR T cells.

In some embodiments, the LEF1⁺ and/or CCR7⁺ and/or TCF1⁺ anti-BCMA CAR T cells are LEF1⁺ CCR7⁺TCF1⁺CD27⁺ anti-BCMA CAR T cells.

In some embodiments, the CD27⁺and/or LEF1⁺ and/or CCR7⁺ and TCF1⁺ anti-BCMA CAR T cells comprise CD4⁺ anti-BCMA CAR T cells.

In particular embodiments, the CD27⁺ and/or LEF1⁺ and/or CCR7⁺ and TCF1⁺ anti-BCMA CAR T cells comprise CD8⁺ anti-BCMA CAR T cells.

In particular embodiments, the CD27⁺ and/or LEF1⁺ and/or CCR7⁺ and TCF1⁺ anti-BCMA CAR T cells comprise CD4⁺ and CD8⁺ anti-BCMA CAR T cells.

In certain embodiments, the cells were manufactured from a subject that has a multiple myeloma or a lymphoma.

In particular embodiments, the cells were manufactured from a subject that has relapsed/refractory multiple myeloma.

In some embodiments, the cells comprise a lentivirus comprising a polynucleotide encoding the anti-BCMA CAR.

In particular embodiments, the anti-BCMA CAR comprises the amino acid sequence set forth in SEQ ID NO: 1.

In further embodiments, the anti-BCMA CAR is encoded by a polynucleotide sequence set forth in SEQ ID NO: 2.

In particular embodiments, the cells are autologous.

In certain embodiments, the cells are cryopreserved.

In particular embodiments, the cells are formulated for administration to a subject that has multiple myeloma or lymphoma.

In some embodiments, human anti-B cell maturation antigen (BCMA) chimeric antigen receptor (CAR) T cells that have been contacted ex vivo with a phosphatidyl-inositol-3 kinase (PI3K) inhibitor for about 5 to about 7 days are provided, wherein the gene expression of 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold or at least 2-fold greater in the anti-BCMA CAR T cells than in an anti-BCMA CAR T cells contacted ex vivo with the PI3K inhibitor for about 10 days.

In particular embodiments, human anti-B cell maturation antigen (BCMA) chimeric antigen receptor (CAR) T cells that have been contacted ex vivo with a phosphatidyl-inositol-3 kinase (PI3K) inhibitor for about 5 to about 7 days are provided, wherein the gene expression of 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQ01 is at least 1.5-fold or at least 2-fold less in the anti-BCMA CAR T cells than in an anti-BCMA CAR T cells contacted ex vivo with the PI3K inhibitor for about 10 days.

In further embodiments, human anti-B cell maturation antigen (BCMA) chimeric antigen receptor (CAR) T cells that have been contacted ex vivo with a phosphatidyl-inositol-3 kinase (PI3K) inhibitor for about 5 to about 7 days are provided; wherein the gene expression of each of 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold or at least 2-fold greater and the gene expression 1, 2, 3, 4, 5, 6, 7, 8, 9, or all of (i) NQ01, CCNA1, IL17F, EMP1, SNHG19, PRR 22, ILDR2, ATAD3, NKD2 and WDR62 or (ii) NKD2 and NQO1 is at least 1.5-fold or at least 2-fold less, in the anti-BCMA CAR T cells than in an anti-BCMA CAR T cells contacted ex vivo with the PI3K inhibitor for about 10 days.

In particular embodiments, CD4⁺ anti-BCMA CAR T cells have a central memory T cell (TCM) like phenotype.

In further embodiments, CD8⁺ anti-BCMA CAR T cells have a stem cell memory T cell (TSCM) like phenotype.

In particular embodiments, CD4+ anti-BCMA CAR T cells have a TCM like phenotype and CD8⁺ anti-BCMA CAR T cells have a TSCM like phenotype.

In some embodiments, the cells were manufactured from a subject that has a multiple myeloma or a lymphoma.

In certain embodiments, the cells were manufactured from a subject has relapsed/refractory multiple myeloma.

In particular embodiments, the cells comprise a lentivirus comprising a polynucleotide encoding the anti-BCMA CAR.

In particular embodiments, the anti-BCMA CAR comprises the amino acid sequence set forth in SEQ ID NO: 1.

In particular embodiments, the anti-BCMA CAR is encoded by a polynucleotide sequence set forth in SEQ ID NO: 2.

In certain embodiments, the cells are autologous.

In certain embodiments, the cells are cryopreserved.

In particular embodiments, the cells are formulated for administration to a subject that has multiple myeloma or lymphoma.

In further embodiments, the PI3K inhibitor is ZSTK474.

In particular embodiments, a pharmaceutical composition comprising a physiologically acceptable excipient and a therapeutically effective amount of the anti-BCMA CAR T cells contemplated herein is provided.

In some embodiments, the therapeutically effective amount of the anti-BCMA CAR T cells is at least about 5.0×10⁷ anti-BCMA CAR T cells.

In certain embodiments, the therapeutically effective amount of the anti-BCMA CAR T cells is at least about 15.0×10⁷ anti-BCMA CAR T cells.

In particular embodiments, wherein the therapeutically effective amount is at least about 45.0×10⁷ anti-BCMA CAR T cells.

In particular embodiments, the therapeutically effective amount is at least about 80.0×10⁷ anti-BCMA CAR T cells.

In further embodiments, the composition is formulated in a solution comprising 50:50 PlasmaLyte A to CryoStor CS10.

In particular embodiments, a method of treating a subject that has multiple myeloma or lymphoma with a composition contemplated herein is provided.

In certain embodiments, the subject has relapsed/refractory multiple myeloma.

In various embodiments, a method for manufacturing anti-BCMA CAR T cells is provided comprising: activating a population of T cells and stimulating the population of T cells to proliferate; transducing the T cells with a lentiviral vector encoding an anti-BCMA CAR that comprises the amino acid sequence set forth in SEQ ID NO: 1; culturing the transduced T cells to proliferate for a period of about 5 to about 7 days; wherein the previous steps are performed in the presence of a PI3K inhibitor, and wherein the gene expression of 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold or at least two-fold greater in the cultured T cells compared to T cells transduced with a lentiviral vector encoding an anti-BCMA CAR that comprises the amino acid sequence set forth in SEQ ID NO: 1 and cultured to proliferate for a period of about 10 days.

In particular embodiments, a method for manufacturing anti-BCMA CAR T cells is provided comprising: activating a population of T cells and stimulating the population of T cells to proliferate; transducing the T cells with a lentiviral vector encoding an anti-BCMA CAR that comprises the amino acid sequence set forth in SEQ ID NO: 1; culturing the transduced T cells to proliferate for a period of about 5 to about 7 days; wherein the foregoing steps are performed in the presence of a PI3K inhibitor, and wherein the gene expression of 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQO1 is at least 1.5-fold or at least two-fold less in the cultured T cells compared to T cells transduced with a lentiviral vector encoding an anti-BCMA CAR that comprises the amino acid sequence set forth in SEQ ID NO: 1 and cultured to proliferate for a period of about 10 days.

In various embodiments, a method for manufacturing anti-BCMA CAR T cells is provided comprising: activating a population of T cells and stimulating the population of T cells to proliferate; transducing the T cells with a lentiviral vector encoding an anti-BCMA CAR that comprises the amino acid sequence set forth in SEQ ID NO: 1; culturing the transduced T cells to proliferate for a period of about 5 to about 7 days; wherein the foregoing steps are performed in the presence of PI3K inhibitor, and wherein the gene expression of 1, 2, 3, 4, 5, 6, 7, 8, 9, 10, or all of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B is at least 1.5-fold or at least two-fold greater and the gene expression of 1, 2, 3, 4, 5, 6, 7, 8, 9, or all of (i)NQ01, CCNA1, IL17F, EMP1, SNHG19, PRR 22, ILDR2, ATAD3, NKD2 and WDR62 or (ii) NKD2 and NQO1 is at least 1.5-fold or at least two-fold less, in the cultured T cells compared to T cells transduced with a lentiviral vector encoding an anti-BCMA CAR that comprises the amino acid sequence set forth in SEQ ID NO: 1 and cultured to proliferate for a period of about 10 days.

In various embodiments, a method for manufacturing anti-BCMA CAR T cells is provided comprising: activating a population of T cells and stimulating the population of T cells to proliferate; transducing the T cells with a lentiviral vector encoding an anti-BCMA CAR that comprises the amino acid sequence set forth in SEQ ID NO: 1; culturing the transduced T cells to proliferate for a period of about 5 to about 7 days; wherein the foregoing steps are performed in the presence of PI3K inhibitor, and wherein the proliferated cells are CD27⁺ and/or LEF1⁺ and/or CCR7⁺ and/or TCF1⁺.

In particular embodiments, the anti-BCMA CAR T cells comprise at least 10% CD27⁺ and/or LEF1⁺ and/or CCR7⁺ and/or TCF1¹⁺ cells.

In further embodiments, the anti-BCMA CAR T cells comprise at least 15% CD27⁺ and/or LEF1⁺ and/or CCR7⁺ and/or TCF1⁺ cells.

In some embodiments, the anti-BCMA CAR T cells comprise at least 20% CD27⁺ and/or LEF1⁺ and/or CCR7⁺ and/or TCF1⁺ cells.

In some embodiments, the anti-BCMA CAR T cells comprise at least 25% CD27⁺ and/or LEF1⁺ and/or CCR7⁺ and/or TCF1⁺T cells.

In particular embodiments, the anti-BCMA CAR T cells comprise at least 30% CD27⁺ and/or LEF1⁺ and/or CCR7⁺ and/or TCF1⁺ cells.

In additional embodiments, the CD27⁺ cells are LEF1⁺ and/or CCR7⁺ and/or TCF1⁺.

In further embodiments, the CD27⁺ cells are LEF1⁺ and CCR7⁺ and TCF1⁺.

In particular embodiments, the CD27⁺ and/or LEF1⁺ and/or CCR7⁺ and/or TCF1⁺ anti-BCMA CAR T cells comprise CD4⁺ anti-BCMA CAR T cells.

In certain embodiments, the CD27⁺ and/or LEFl⁺ and/or CCR7⁺ and/or TCF l⁺ anti-BCMA CAR T cells comprise CD8⁺ anti-BCMA CAR T cells.

In additional embodiments, the CD27⁺ and/or LEF1⁺ and/or CCR7⁺ and/or TCF1⁺ anti-BCMA CAR T cells comprise CD4⁺ and CD8⁺ anti-BCMA CAR T cells.

In particular embodiments, the T cells are autologous.

In additional embodiments, the method further comprises isolating peripheral blood mononuclear cells (PBMCs) as the source of T cells.

In some embodiments, the PBMCs are isolated from a subject that has a multiple myeloma or a lymphoma.

In certain embodiments, the subject has relapsed/refractory multiple myeloma.

In particular embodiments, the method further comprises cryopreserving the PBMCs before activation and stimulation.

In further embodiments, the T cells are cryopreserved expansion culture.

In further embodiments, the T cell are activated and simulated to proliferate for about 18 to about 24 hours.

In certain embodiments, activation of the T cells comprises contacting the T cells with an anti-CD3 antibody or antigen binding fragment thereof.

In particular embodiments, the anti-CD3 antibody or antigen binding fragment thereof is soluble.

In additional embodiments, the anti-CD3 antibody or antigen binding fragment thereof is bound to a surface.

In some embodiments, the surface is a bead, optionally a paramagnetic bead.

In further embodiments, stimulation of the T cells comprises contacting the T cells with an anti-CD28 antibody or antigen binding fragment thereof.

In particular embodiments, the anti-CD28 antibody or antigen binding fragment thereof is soluble.

In additional embodiments, the anti-CD28 antibody or antigen binding fragment thereof is bound to a surface.

In some embodiments, the surface is a bead, optionally a paramagnetic bead, optionally the paramagnetic bead bound to the anti-CD3 antibody or antigen binding fragment thereof.

In particular embodiments, the cells are transduced with an HIV-1 derived lentiviral vector.

In some embodiments, the anti-BCMA CAR is encoded by the polynucleotide sequence set forth in SEQ ID NO: 2.

In further embodiments, the PI3K inhibitor is ZSTK474.

In various embodiments, a method for increasing CD4⁺ TCM like anti-BCMA CAR T cells and CD8⁺ TSCM like anti-BCMA CAR T cells in an adoptive cell therapy is provided comprising contacting anti-BCMA CAR T cells ex vivo with a PI3K inhibitor for about 5 to about 7 days, wherein the number of CD4⁺ TCM like anti-BCMA CAR T cells and CD8⁺ TSCM like anti-BCMA CAR T cells is at least two-fold greater in the anti-BCMA CAR T cells than in anti-BCMA CAR T cells contacted ex vivo with the PI3K inhibitor for about 10 days.

In certain embodiments, the anti-BCMA CAR T cells comprise at least 10% CD27⁺ and/or LEF1⁺ and/or CCR7⁺ and/or TCF1⁺ T cells.

In additional embodiments, the anti-BCMA CAR T cells comprise at least 15% CD27⁺ and/or LEF1⁺ and/or CCR7⁺ and/or TCF1⁺T cells.

In some embodiments, the anti-BCMA CAR T cells comprise at least 20% CD27⁺ and/or LEF1⁺ and/or CCR7⁺ and/or TCF1⁺T cells.

In particular embodiments, the anti-BCMA CAR T cells comprise at least 25% CD27⁺ and/or LEF1⁺ and/or CCR7⁺ and/or TCF1⁺T cells.

In further embodiments, the anti-BCMA CAR T cells comprise at least 30% CD27⁺ and/or LEF1⁺ and/or CCR7⁺ and/or TCF1⁺T cells.

In certain embodiments, the T cells are autologous.

In particular embodiments, the method further comprises isolating peripheral blood mononuclear cells (PBMCs) as the source of T cells.

In additional embodiments, the PBMCs are isolated from a subject that has a multiple myeloma or a lymphoma.

In some embodiments, the subject has relapsed/refractory multiple myeloma.

In further embodiments, the anti-BCMA CAR T cells comprise an HIV-1 derived lentiviral vector.

In particular embodiments, the anti-BCMA CAR comprises the amino acid sequence set forth in SEQ ID NO: 1.

In additional embodiments, the anti-BCMA CAR is encoded by the polynucleotide sequence set forth in SEQ ID NO: 2.

In some embodiments, a pharmaceutical composition comprising a pharmaceutically acceptable excipient and a therapeutically effective amount of the anti-BCMA CAR T cells contemplated herein is provided.

In certain embodiments, a pharmaceutical composition comprising a pharmaceutically acceptable excipient and a therapeutically effective amount of the CD4⁺ TCM anti-BCMA CAR T cells and CD8⁺ TSCM anti-BCMA CAR T cells contemplated herein is provided.

In particular embodiments, a method of treating a subject that has multiple myeloma or lymphoma comprises administering a composition contemplated herein.

In further embodiments, the subject has relapsed/refractory multiple myeloma.

In various embodiments, a method for increasing the gene expression of each of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B in anti-BCMA CAR T cells is provided comprising contacting anti-BCMA CAR T cells ex vivo with a PI3K inhibitor for about 5 to about 7 days, wherein the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater in the anti-BCMA CAR T cells than in anti-BCMA CAR T cells contacted ex vivo with the PI3K inhibitor for about 10 days.

In particular embodiments, a method for decreasing the gene expression of each of (i) NQO1, CCNA1, IL17F, EMP1, SNHG19, PRR 22, ILDR2, ATAD3, NKD2 and WDR62 or (ii) NKD2 and NQO1 in anti-BCMA CAR T cells is provided comprising contacting anti-BCMA CAR T cells ex vivo with a PI3K inhibitor for about 5 to about 7 days, wherein the gene expression of each of (i) NQO1, CCNA1, IL17F, EMP1, SNHG19, PRR 22, ILDR2, ATAD3, NKD2 and WDR62 or (ii) NKD2 and NQO1 is at least 1.5-fold less in the anti-BCMA CAR T cells than in anti-BCMA CAR T cells contacted ex vivo with the PI3K inhibitor for about 10 days.

In certain embodiments, a method for increasing the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNT5B and decreasing the gene expression of each of (i) NQO1, CCNA1, IL17F, EMP1, SNHG19, PRR 22, ILDR2, ATAD3, NKD2 and WDR62 or (ii) NKD2 and NQ01 in anti-BCMA CAR T cells is provided comprising contacting anti-BCMA CAR T cells ex vivo with a PI3K inhibitor for about 5 to about 7 days, wherein the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater and the gene expression of each of (i) NQ01, CCNA1, IL17F, EMP1, SNHG19, PRR 22, ILDR2, ATAD3, NKD2 and WDR62 or (ii) NKD2 and NQO1 is at least 1.5-fold less, in the anti-BCMA CAR T cells than in anti-BCMA CAR T cells contacted ex vivo with the PI3K inhibitor for about 10 days.

In some embodiments, a method for increasing the therapeutic efficacy of anti-BCMA CAR T cells is provided comprising contacting anti-BCMA CAR T cells ex vivo with a PI3K inhibitor for about 5 to about 7 days, wherein the increase in therapeutic efficacy is indicated by an increase in gene expression of each of 1, 2. 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater in the anti-BCMA CAR T cells compared to anti-BCMA CAR T cells contacted ex vivo with the PI3K inhibitor for about 10 days.

In particular embodiments, a method for increasing the therapeutic efficacy of anti-BCMA CAR T cells is provided comprising contacting anti-BCMA CAR T cells ex vivo with a PI3K inhibitor for about 5 to about 7 days, wherein the increase in therapeutic efficacy is indicated by a decrease in gene expression of each of (i) NQ01, CCNA1, IL17F, EMP1, SNHG19, PRR 22, ILDR2, ATAD3, NKD2 and WDR62 or (ii) NKD2 and NQ01 is at least 1.5-fold less in the anti-BCMA CAR T cells compared to anti-BCMA CAR T cells contacted ex vivo with the PI3K inhibitor for about 10 days.

In some embodiments, method for increasing the therapeutic efficacy of anti-BCMA CAR T cells is provided comprising contacting anti-BCMA CAR T cells ex vivo with a PI3K inhibitor for about 5 to about 7 days, wherein the increase in therapeutic efficacy is indicated by an increase in gene expression of each 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater and a decrease in gene expression of each of (i) NQO1, CCNA1, IL17F, EMP1, SNHG19, PRR 22, ILDR2, ATAD3, NKD2 and WDR62 or (ii) NKD2 and NQ01 is at least 1.5-fold less, in the anti-BCMA CAR T cells compared to anti-BCMA CAR T cells contacted ex vivo with the PI3K inhibitor for about 10 days.

In particular embodiments, the anti-BCMA CAR T cells are from a subject that has a multiple myeloma or a lymphoma.

In additional embodiments, the anti-BCMA CAR T cells are from a subject has relapsed/refractory multiple myeloma.

In certain embodiments, the anti-BCMA CAR T cells comprises an HIV-1 derived lentiviral vector comprising a polynucleotide encoding the anti-BCMA CAR.

In particular embodiments, the anti-BCMA CAR comprises the amino acid sequence set forth in SEQ ID NO: 1.

In further embodiments, the anti-BCMA CAR is encoded by a polynucleotide sequence set forth in SEQ ID NO: 2.

In some embodiments, the anti-BCMA CAR T cells are autologous. In particular embodiments, the PI3K inhibitor is ZSTK474.

### BRIEF DESCRIPTION OF THE SEVERAL VIEWS OF THE DRAWINGS

FIG. 1 shows that the length of T cell culture with PI3K inhibitor modulates T cell phenotype. Five multiple myeloma PBMC lots were used to manufacture anti-BCMA CAR T cells in the absence of PI3K inhibitor or cultured with PI3K inhibitor for 7 days or 10 days post-transduction with a lentivirus encoding an anti-BCMA CAR. T cells were stained at day 7 and day 10 with anti-human antibodies against CD3, CD62L, CCR7, and CD45RA and analyzed by flow cytometry. Each dot plot was gated on viable CD3⁺ lymphocytes.

FIG. 2 shows that T cells show a more potent phenotype after 7 days of culture with PI3K inhibitor compared to 10 days of culture. Five multiple myeloma PBMC lots were used to manufacture anti-BCMA CAR T cells in the presence of PI3K inhibitor for 7 days or 10 days. T cells were stained at day 7 and day 10 with anti-human antibodies against CCR7, CD25, CD28, CD122, ICOS, CD45RO, CD57, and TIM3 and analyzed by CyTOF. Each dot plot was gated on viable CD3⁺ lymphocytes.

FIG. 3 shows T cells manufactured for 7 days in PI3K inhibitor are enriched in CD27⁺ T cells. Five multiple myeloma PBMC lots were used to manufacture anti-BCMA CAR T cells in the presence of PI3K inhibitor. T cells were stained at day 7 and day 10 with anti-human antibodies against CD4, CD8, and CD27 and analyzed by CyTOF. VISNE plots show CD27 gated expression in different cell populations.

FIGS. 4A-B show that T cells show a more potent phenotype after 7 days of culture with PI3K inhibitor compared to 10 days of culture. Five multiple myeloma PBMC lots were used to manufacture anti-BCMA CAR T cells in the presence of PI3K inhibitor for 7 days or 10 days. T cells were stained at day 7 and day 10 with anti-human antibodies against (1) CCR7, CD25, CD28, HLA-DR, and TIM3 (FIG. 4A) or, CD45RO, CD57, CD70, CD244, and PD-1 (FIG. 4B) and analyzed by CyTOF. VISNE plots show expression of different T cell phenotypic markers in the 7 day culture (top row) and the 10 day culture (bottom row). Gated population represents CD27⁺ cells.

FIG. 5 shows CD27⁺ T cells manufactured for 10 days in PI3K inhibitor are marked by decreased activation and increased exhaustion compared to T cells manufactured for 7 days in PI3K inhibitor. Five multiple myeloma PBMC lots were used to manufacture anti-BCMA CAR T cells in the presence of PI3K inhibitor for 7 days or 10 days. CD27⁺ T cells identified by VISNE analysis were stained at day 7 and day 10 with anti-human antibodies against CD28, ICOS, HLA-DR, CD25, and TIM3 and analyzed by CyTOF in CD4+ T cells (top) and CD8⁺ T cells (bottom).

FIG. 6 shows differential gene expression as a result of the duration of anti-BCMA CAR T cell manufacturing. Multiple myeloma PBMC lots were used to manufacture anti-BCMA CAR T cells in the absence of PI3K inhibitor for 7 days (1) or 10 days (13) or presence of PI3K inhibitor for 7 days (10) or 10 days (6). RNA was extracted from T cells and the transcriptional profile was analyzed using a Nanostring Immunology panel. A heatmap of the top 50 differentially expressed genes between manufacturing conditions is shown.

FIG. 7 shows the increased potency of anti-BCMA CAR T cells manufactured for 7 days in PI3K inhibitor compared to anti-BCMA CAR T cells manufactured for 10 days in PI3K inhibitor. Healthy donor PBMCs were activated, transduced with a lentiviral vector encoding an anti-BCMA CAR, and expanded in the presence of IL-2 and PI3K inhibitor for 6 days (7 day process) or 9 days (10 day process). NSG mice were injected intravenously with 2×10⁶ firefly luciferase labeled Daudi tumor cells 10 days before adoptive cell therapy. Mice were injected with 2.5, 5 or 10×10⁶ anti-BCMA CAR^{T} T cells or T cells transduced with vehicle. The tumor burden was monitored by luminescence.

FIG. 8 shows that T cells manufactured in the presence of PI3K are enriched for CD27⁺ CD4⁺ TCM like cells and CD27⁺ CD8⁺ TSCM like cells. Anti-BCMA CAR T cells manufactured from multiple myeloma PBMC lots in the presence of PI3K inhibitor were stained with a panel of ˜36 T cell phenotyping antibodies and analyzed with CyTOF. Naïve T cells (Tₙₐᵢᵥₑ), Central memory T cells (TCM), Effector memory T cells (EM), Effector T cells (TEff), and Stem cell memory T cells (TSCM) are shown. The data presented shows each DP lot analyzed as a function of the % of CD27⁺ enriched cells vs. T cell subset.

FIG. 9 shows the CD8⁺ T cell data from FIG. 9 analyzed using FlowSOM. FlowSOM identified 20 distinct T cell clusters. Three major groups of T cells were identified based on clusters 4 (enriched in memory T cell markers—favorable) and cluster 5 (enriched in effector T cell markers—less favorable). %CD27⁺CD8⁺ T cells, manufacturing method, and clinical responses for subjects treated with the anti-BCMA CAR T cells are shown.

FIG. 10 shows differential gene expression analysis of anti-BCMA CAR T cells manufactured from multiple myeloma cell lots using a 7 day or 10 day PI3K manufacturing process. RNA was extracted from 12 lots of anti-BCMA CAR T cells and the transcriptional profile was analyzed using a Nanostring Immunology panel. A heatmap of the top 25 differentially expressed genes between the 7 day and 10 day manufacturing processes is shown. %CD27⁺ T cells, manufacturing method, and clinical responses for subjects treated with the anti-BCMA CAR T cells are shown.

FIG. 11A shows a volcano plot for cyTOF stained T cell populations in anti-BCMA CAR T cell drug products in durable compared to nondurable responders. The plot shows that the most significant differences in cell composition between durable and nondurable responders are naive and stem cell memory T cells. The generalized linear model coefficient is shown on the X axis and the p-value on the Y axis.

FIG. 11B shows box plots of the proportion of CD4 TSCM (top panel) and CD8 TSCM (bottom panel) in anti-BCMA CAR T cell drug products compared to durable and nondurable responders. TSCM cells were enriched in the drug products of patients with durable responses.

FIG. 12A shows box plots of the proportion of LEF-1 expression determined by CyTOF in CD4 (top left panel) and CD8 (top center panel) T cells in anti-BCMA CAR T cell drug products compared to durable and nondurable responders. The proportion of LEF-1 expressing cells as well as the gene expression of LEF-1 are increased in durable compared to nondurable responders indicates an enrichment for early memory T cells in these drug products.

FIG. 12A shows the correlation of LEF-1 gene expression in drug products with patient sBCMA levels two months after treatment with anti-BCMA CAR T cells. These data indicate an association between early memory phenotype in the drug product and depth of treatment response.

FIG. 13 shows the percentage of CD3+ live cells expressing CCR7 (top left panel), LEF1 (top center panel) and CD57 (top right panel) determined by CyTOF in PBMC and in anti-BCMA CAR T cells (DP). FIG. 13 further shows the percentage of CD3+ live cells expressing CCR7 (FIG. 13, bottom left panel), LEF-1 (FIG. 13, bottom center panel) and CD57 (FIG. 13, bottom right panel) on they axis compared to the maximum vector copy number (VCN) determined by PCR on CD3+ cells extracted from whole blood at various time points after infusion of anti-BCMA CAR T cells on the x axis.

FIG. 14 shows the percentage of CD3+ live cells expressing CD57 (marker of senescence), LEF-1, CCR7 and CD27 (memory cells) as a clustered heatmap. The data were grouped using average linkage hierarchical clustering and the top 3 clusters as determined by the cluster dendrograms were associated with patients' clinical response at 6 months.

### BRIEF DESCRIPTION OF THE SEQUENCE IDENTIFIERS

SEQ ID NO: 1 sets forth the amino acid sequence of an anti-BCMA CAR.

SEQ ID NO: 2 sets forth the polynucleotide sequence encoding an anti-BCMA CAR.

In the foregoing sequences, X, if present, refers to any amino acid or the absence of an amino acid.

### DETAILED DESCRIPTION

### A. Overview

The invention generally relates to improved methods for manufacturing T cell compositions. Although T cell therapies are more prevalent than they were 5 years ago, the obstacles faced by these therapies still remain, notably, poor or suboptimal potency. The solution is provided by the present manufacturing methods, which vastly increase the potency of cell therapy products, e.g., CAR T cell products. Without wishing to be bound to any particular theory, the inventors have unexpectedly discovered that decreasing the duration of T cell manufacturing using a PI3K inhibitor enables further improvements in reducing cell dose and increasing cell potency and persistence compared to longer duration manufacturing processes using the PI3K inhibitor. Surprisingly, the improved drug products manufactured using a shorter PI3K inhibitor-based process have enriched populations of CD27⁺CD8⁺ stem cell memory T cells (TSCM) and CD27⁺CD4⁺ central memory T cells (TCM). In particular embodiments, the improved drug products manufactured using a shorter PI3K inhibitor-based process have enriched populations of CD27⁺, LEF1⁺, and/or TCF1⁺T cells. The manufactured cells are able to subsequently differentiate and provide durable immune effector cell function.

Drug product phenotyping and gene expression analysis also enables the clinician to determine the likelihood of how a particular drug product may perform. The enriched T cells also comprise increased gene expression of one or more of: Nuclear Receptor Subfamily 4 Group A Member 2 (NR4A2), CD229 (LY9), Lin-7 Homolog A (LIN7A), Wingless-Type MMTV Integration Site Family, Member 5B (WNT5B), B cell CLL/lymphoma 6 (BCL6), Early Growth Response 1 (EGR1), Early Growth Response 2 (EGR2), Activating Transcription Factor 3 (ATF3), C-C motif chemokine 1 (CCL1), Interleukin lA (IL-1A), and C-C motif chemokine 5 (CCL5); and decreased gene expression of one or more of: NAD(P)H Quinone Dehydrogenase 1 (NQO1), Cyclin Al (CCNA1), Interkleukin 17F (IL17F), Epithelial Membrane Protein 1 (EMP1), Small Nucleolar RNA Host Gene 19 (SNHG19), Proline Rich 22 (PRR 22), Immunoglobulin Like Domain Containing Receptor 2 (ILDR2), ATPase Family, AAA Domain Containing 3 (ATAD3), Naked Cuticle Homolog 2 (NKD2) and WD Repeat Domain 62 (WDR62).

In particular embodiments, the enriched T cells comprise increased gene expression of one or more of: CCL1, NR4A2, ATF3, CCL5, and WNT5B; and decreased gene expression of one or more of: NQO1 and NKD2.

In various embodiments, a method for manufacturing T cells is provided that increases the potency of adoptive cell therapies is provided. In particular preferred embodiments, an engineered CAR T cell composition is manufactured in the presence of a phosphatidyl-inositol-3 kinase (PI3K) inhibitor (e.g., ZSTK474 (CAS NO. 475110-96-4)) for a duration an under conditions sufficient to increase the potency of the engineered cells. In preferred embodiments, T cells are activated and stimulated in the presence of a PI3K inhibitor (about 24 hours, 18-24 hours), transduced with a lentivirus comprising a polynucleotide that encodes a CAR in the presence of the PI3K inhibitor(about 24 hours, 18-24 hours), and expanded in the presence of a PI3K inhibitor for about 4 days or about 6 days post-transduction (e.g., 6 days or 8 days total, resp.).

In various embodiments, a five day T cell manufacturing process activating and stimulating T cells in the presence of a PI3K inhibitor (about 24 hours, 18-24 hours), transducing the cells with a lentivirus comprising a polynucleotide that encodes a CAR in the presence of the PI3K inhibitor(about 24 hours, 18-24 hours), and expanding the cells in the presence of a PI3K inhibitor for about 4 days (e.g., 6 days total).

In various embodiments, a seven day T cell manufacturing process activating and stimulating T cells in the presence of a PI3K inhibitor (about 24 hours, 18-24 hours), transducing the cells with a lentivirus comprising a polynucleotide that encodes a CAR in the presence of the PI3K inhibitor(about 24 hours, 18-24 hours), and expanding the cells in the presence of a PI3K inhibitor for about 6 days (e.g., 8 days total).

In particular embodiments, methods of increasing the expression T cell activation or potency genes and/or decreasing expression of T cell differentiation or exhaustion genes is contemplated. Manufactured T cell compositions contemplated herein are useful in the treatment of, prevention of, or amelioration of at least one symptom of a cancer, e.g., a hematological malignancy.

In various embodiments, current Good Manufacturing Practice (cGMP) manufactured compositions of CD27⁺ enriched anti-B cell maturation antigen (BCMA) chimeric antigen receptor (CAR) T cells manufactured in the presence of a PI3K inhibitor are contemplated. In particular embodiments, the shorter 5 day or 7 day manufacturing processes generate enriched populations of CD27⁺, LEF1⁺, CCR7⁺ and/or TCF1⁺ anti-BCMA CAR T cells.

In various embodiments, anti-BCMA CAR T cell compositions of CD27^{TCF}1⁺ T enriched CD8^{TCF}1⁺ T TSCM like T cells and CD27⁺ enriched CD4⁺ TCM like T cells are contemplated.

In various embodiments, current Good Manufacturing Practice (cGMP) manufactured compositions of LEF1⁺ and/or CCR7⁺ and/or TCF1⁺ enriched anti-B cell maturation antigen (BCMA) chimeric antigen receptor (CAR) T cells manufactured in the presence of a PI3K inhibitor are contemplated. In particular embodiments, the enriched populations are also CD27⁺ anti-BCMA CAR T cells.

In various embodiments, anti-BCMA CAR T cell compositions of CD27⁺ and/or LEF1⁺ and/or CCR7⁺ and/or TCF1⁺ enriched CD8⁺ TSCM like T cells and CD27⁺ and/or LEF1⁺ and/or CCR7⁺ and/or TCF1⁺ enriched CD8⁺ TCM like T cells are contemplated.

Accordingly, the methods and compositions contemplated herein represent a quantum improvement compared to existing adoptive cell immunotherapies.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in joumals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “substantially” refers to a quantity, level, value, number, frequency, percentage, dimension, size, amount, weight or length that is 80%, 85%, 90%, 91%, 92%, 93%, 94%, 95%. 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “T cell manufacturing” or “methods of manufacturing T cells” or comparable terms refer to the process of producing a therapeutic composition of T cells, which manufacturing methods may comprise one or more of, or all of the following steps: harvesting, stimulation, activation, transduction, and expansion. In preferred embodiments, expansion is no more than 5 days to 7 days, post-transduction. A five day T cell manufacturing process comprises activation and stimulation at Day 0, transduction at Day 1, and expansion until the end of Day 5. A seven day T cell manufacturing process comprises activation and stimulation at Day 0, transduction at Day 1, and expansion until the end of Day 7. A 10 day T cell manufacturing process comprises activation and stimulation at Day 0, transduction at Day 1, and expansion until the end of Day 10. In preferred embodiments, T cell manufacturing methods comprise the use of a PI3K throughout the manufacturing process.

As used herein, the term “PI3K inhibitor” refers to a small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p1106δ, and p110γ) and one of two families of regulatory subunits. In particular embodiments, a PI3K inhibitor displays selectivity for one or more isoforms of the class 1 PI3K inhibitors (i.e., selectivity for p110α, p110β, p1106δ, and p110γ or one or more of p110α, p110β, p1106δ, and p110γ). In particular embodiments, a PI3K inhibitor will not display isoform selectivity and be considered a “pan-PI3K inhibitor.”

The terms “T cell” or “T lymphocyte” are art-recognized and are intended to include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, CD4⁺CD8⁺T cell, or any other subset of T cells. Preferably, manufactured T cells are enriched in CD27⁺ T cells, CD27⁺CD4⁺ T cells and/or CD27⁺CD8⁺ T cells. In a particular preferred embodiment, the manufactured T cells are enriched in LEF1⁺ and/or CCR7⁺ and/or TCF1⁺T cells and/or LEF1⁺ and/or CCR7⁺and/or TCF1⁺ CD4⁺ T cells and/or LEF1⁺ and/or CCR7⁺ and/or TCF1⁺ CD8⁺ T cells. In a particular preferred embodiment, the manufactured T cells are enriched in CD27⁺ LEF1⁺ and/or CCR7⁺ and/or TCF1⁺T cells and/or CD27⁺LEF1⁺ and/or CCR7⁺ and/or TCF1⁺ CD4⁺ T cells and/or CD27⁺ LEF1⁺ and/or CCR7⁺ and/or TCF1⁺ CD8⁺ T cells. More preferably, manufactured T cells are enriched in Stem cell memory T cells (TSCM) and Central memory T cells (TCM).

“Potent T cells,” and “young T cells,” are used interchangeably in particular embodiments and refer to T cell phenotypes wherein the T cell is capable of proliferation and a concomitant decrease in differentiation. In particular embodiments, the young T cell has the phenotype of a naïve T cell TSCM, or TCM. In various embodiments, the manufacturing methods contemplated herein produce more potent T cells, e.g., naïve T cells, TSCMs, or TCMs. In particular embodiments, young T cells comprise are enriched for one or more of, or all of the following biological markers: CD62L, CCR7, CD28, CD27, CD122, CD127, CD197, CD95, CD45RO, and CD38.

As used herein, the term “proliferation” refers to an increase in cell division, either symmetric or asymmetric division of cells. In particular embodiments, “proliferation” refers to the symmetric or asymmetric division of T cells. “Increased proliferation” occurs when there is an increase in the number of cells in a treated sample compared to cells in a non-treated sample.

As used herein, the term “differentiation” refers to a method of decreasing the potency or proliferation of a cell or moving the cell to a more developmentally restricted state. In particular embodiments, differentiated T cells acquire immune effector cell functions.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8⁺ T cells), TILs, and helper T cells (HTLs; CD4+ T cells).

“Modified T cells” refer to T cells that have been modified by the introduction of a polynucleotide encoding a CAR contemplated herein. Modified T cells include both genetic and non-genetic modifications (e.g., episomal or extrachromosomal).

As used herein, the term “genetically engineered” or “genetically modified” refers to the addition of extra genetic material in the form of DNA or RNA into the total genetic material in a cell.

The terms, “genetically modified cells,” “modified cells,” and, “redirected cells,” are used interchangeably.

As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a TCR or CAR and/or one or more cytokines. In particular embodiments, T cells are modified to express a CAR without modifying the genome of the cells, e.g., by introducing an episomal vector that expresses the TCR or CAR into the cell.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism, such as cell self-renewal and expansion of cells. In one embodiment, the term “in vivo expansion” refers to the ability of a cell population to increase in number in vivo.

The term “stimulation” refers to a primary response induced by binding of a stimulatory molecule (e.g., a TCR/CD3 complex) with its cognate ligand thereby mediating a signal transduction event including, but not limited to, signal transduction via the TCR/CD3 complex.

A “stimulatory molecule,” refers to a molecule on a T cell that specifically binds with a cognate stimulatory ligand.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands include, but are not limited to CD3 ligands, e.g., an anti-CD3 antibody and CD2 ligands, e.g., anti-CD2 antibody, and peptides, e.g., CMV, HPV, EBV peptides.

The term, “activation” refers to the state of a T cell that has been sufficiently stimulated to induce detectable cellular proliferation. In particular embodiments, activation can also be associated with induced cytokine production, and detectable effector functions. The term “activated T cells” refers to, among other things, T cells that are proliferating. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the CD3/TCR complex or through CD2.

A “costimulatory signal,” refers to a signal, which in combination with a primary signal, such as TCR/CD3 ligation, leads to T cell proliferation, cytokine production, and/or upregulation or downregulation of particular molecules (e.g., CD28).

A “costimulatory ligand,” refers to a molecule that binds a costimulatory molecule. A costimulatory ligand may be soluble or provided on a surface. A “costimulatory molecule” refers to the cognate binding partner on a T cell that specifically binds with a costimulatory ligand (e.g., anti-CD28 antibody).

“Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells manufactured by the methods contemplated herein are autologous.

As used herein, the terms “individual” and “subject” are often used interchangeably and refer to any animal that exhibits a symptom of a cancer that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

As used herein, the term “patient” refers to a subject that has been diagnosed with a particular indication that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell death killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5.2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or a control composition.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, or a control molecule/composition. A comparable response is one that is not significantly different or measurable different from the reference response.

An “antigen (Ag)” refers to a compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. A “target antigen” or “target antigen or interest” is an antigen that a binding domain of a CAR contemplated herein, is designed to bind.

An “epitope” or “antigenic determinant” refers to the region of an antigen to which a binding agent binds.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full-length protein sequence or a fragment of a full length protein, and may include post-translational modifications of the polypeptide, for example, glycosylations, acetylations, phosphorylations and the like, as well as other modifications known in the art, both naturally occurring and non-naturally occurring. Polypeptides can be prepared using any of a variety of well-known recombinant and/or synthetic techniques. Polypeptides contemplated herein specifically encompass CARs of the present disclosure, or sequences that have deletions from, additions to, and/or substitutions of one or more amino acid of a CAR as disclosed herein. In particular embodiments, the term “polypeptide” further includes variants, fragments, and fusion polypeptides

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. Preferably, polypeptides of the invention include polypeptides having at least about 65%, 70%, 75%, 85%, 90%, 95%, 98%, or 99% amino acid identity thereto.

Polypeptides include “polypeptide fragments.” Polypeptide fragments refer to fragments of a biologically active polypeptide, which can be monomeric or multimeric and that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

Fusion polypeptides and fusion proteins refer to a polypeptide having at least two, three, four, five, six, seven, eight, nine, or ten or more polypeptide segments.

As used herein, the terms “polynucleotide” or “nucleic acid” refers to messenger RNA (mRNA), RNA, genomic RNA (gRNA), plus strand RNA (RNA(⁺)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present invention contemplates, in part, polynucleotides comprising expression vectors, viral vectors, and transfer plasmids, and compositions, and cells comprising the same.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

The term “vector” is used herein to refer to a nucleic acid molecule capable transferring or transporting another nucleic acid molecule.

Additional definitions are set forth throughout this disclosure.

### C. T Cell Manufacturing Methods

The T cells manufactured by the methods contemplated herein provide improved adoptive immunotherapy compositions. The present invention contemplates 5 day to 7 day T cell manufacturing processes using PI3K inhibitors that generate more potent T cells compared to existing 10 day T cell manufacturing processes using such inhibitors. Without wishing to be bound to any particular theory, it is believed that the T cell compositions, e.g., anti-BCMA CAR T cell, manufactured by the methods contemplated herein comprise an increase in the number of (enriched) more potent T cell populations. In particular embodiments, the 5 day to 7 day manufacturing methods contemplated herein result in an enriched population of CD27⁺ and/or LEF1⁺ and/or CCR7⁺ and/or TCF1⁺T cells. In particular embodiments, the 5 day to 7 day manufacturing methods contemplated herein result in an enriched population of CD27⁺ and LEF1⁺ and/or CCR7⁺ and/or TCF1⁺T cells. In particular embodiments, the 5 day to 7 day manufacturing methods contemplated herein result in an enriched population of CD27⁺ and LEF1⁺ and CCR7⁺ and/or TCF1⁺T cells. In particular embodiments, the 5 day to 7 day manufacturing methods contemplated herein result in an enriched population of CD27⁺ and LEF1⁺ and CCR7⁺ and TCF1⁺T cells. In particular embodiments, the 5 day to 7 day manufacturing methods contemplated herein result in an enriched population of CD27⁺CD8⁺ stem cell memory T cells (TSCM) and CD27⁺CD4⁺ central memory T cells (TCM). In particular embodiments, the 5 day to 7 day manufacturing methods contemplated herein result in an enriched population of LEF1⁺CD8⁺ stem cell memory T cells (TSCM) and LEF1⁺CD4⁺ central memory T cells (TCM). In particular embodiments, the 5 day to 7 day manufacturing methods contemplated herein result in an enriched population of CD27⁺LEF1⁺CD8⁺ stem cell memory T cells (TSCM) and CD27⁺LEF1⁺CD4⁺ central memory T cells (TCM). In particular embodiments, the 5 day to 7 day manufacturing methods contemplated herein result in an enriched population of CD27⁺LEF1⁺ CCR7⁺CD8⁺ stem cell memory T cells (TSCM) and CD27⁺LEF1⁺CCR7⁺CD4⁺ central memory T cells (TCM). In particular embodiments, the 5 day to 7 day manufacturing methods contemplated herein result in an enriched population of CD27⁺LEF1⁺CF1⁺CD8⁺ stem cell memory T cells (TSCM) and CD27⁺LEF1⁺TF1⁺CD4⁺ central memory T cells (TCM). In particular embodiments, the 5 day to 7 day manufacturing methods contemplated herein result in an enriched population of CD27⁺LEF1⁺TF1⁺CD8⁺ stem cell memory T cells (TSCM) and CD27⁺LEF1⁺TF1⁺CD4⁺ central memory T cells (TCM). Moreover, the 5 day to 7 day T cell manufacturing processes using PI3K inhibitors comprise differential gene expression signatures compared to T cells manufactured with the 10 day process using the PI3Kinhibitors. Adoptive cell therapies, e.g., CAR T cell therapies, comprising these enriched cell populations allow clinicians to reduce cell dose and increasing cell potency and persistence, without comprising the efficacy of the therapy.

In various embodiments, a method for manufacturing T cells comprises activating a population of T cells and stimulating the population of T cells to proliferate; transducing the T cells with a viral vector comprising a polynucleotide encoding a CAR; and culturing the transduced T cells to proliferate for a period of about 4 days to about 6 days; wherein all the methods steps are performed in the presence of a PI3K inhibitor.

Illustrative examples of PI3K inhibitors suitable for use in particular embodiments of the T cell manufacturing methods contemplated herein include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, p1106δ, and p110γ isoforms, Oncothyreon). Other illustrative examples of selective PI3K inhibitors include, but are not limited to BYL719, GSK2636771, TGX-221, AS25242, CAL-101, ZSTK474, and IPI-145. Further illustrative examples of pan-PI3K inhibitors include, but are not limited to BEZ235, LY294002, GSK1059615, TG100713, and GDC-0941.

In the most preferred embodiments, contemplated herein_{;} the manufacturing methods use the PI3K inhibitor ZSTK474 (CAS NO. 475110-96-4).

In various embodiments, the PI3K inhibitor is used at a concentration of at least 1 nM, at least 2 nM, at least 5 nM, at least 10 nM at least 50 nM, at least 100 nM, at least 200 nM, at least 500 nM, at least 1 μM, at least 10 μM, at least 50 μM, at least 100 μM, or at least 1 M throughout the manufacturing process.

In preferred embodiments, the PI3K inhibitor is used at a concentration of about 1 μM throughout the manufacturing process

T cells can be obtained from a number of sources including, but not limited to, peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In particular embodiments, PBMCs are used as the source of T cells in the T cell manufacturing methods contemplated herein. PBMCs form a heterogeneous population of T lymphocytes that can be CD4⁺, CD8⁺, or CD4⁺ and CD8⁺ and can include other mononuclear cells such as monocytes, B cells, NK cells and NKT cells.

In preferred embodiments, the T cell manufacturing process begins by obtaining a source of PBMCs from the circulating blood of an individual by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media. Methods for T cell manufacturing are disclosed in U.S. patent application Ser. No. 15/306,729, entitled “Improved Methods for Manufacturing Adoptive Cell Therapies,” filed Oct. 25, 2016; U.S. patent application Ser. No. 15/316,792, entitled “Improved T Cell Compositions,” filed Dec. 6, 2016; and U.S. patent application Ser. No 16/060,184, entitled “Improved T Cell Compositions,” filed Jun. 7, 2018, each of which is incorporated herein by reference in its entirety. In particular embodiments, a population of cells comprising T cells, e.g., PBMCs, is used in the manufacturing methods contemplated herein. In other embodiments, an isolated or purified population of T cells is used in the manufacturing methods contemplated herein.

PBMCs may be treated to activate and stimulate T cell populations contained therein to achieve sufficient therapeutic doses of T cell compositions. In particular embodiments, T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In preferred embodiments, T cells are activated and stimulated in the presence of a PI3K inhibitor, e.g., ZSTK474. The methods contemplated here differ from existing methods in that only a single round of activation and stimulation is performed wherein methods in the art routinely use two, three, four, or five or more rounds of activation and expansion.

T cell activation can be accomplished by providing a primary stimulation signal through the T cell TCR/CD3 complex or via stimulation of the CD2 surface protein. The TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1. In addition to the primary stimulation signal provided through the TCR/CD3 complex, or via CD2, induction of T cell responses requires a second, costimulatory signal. In particular embodiments, a CD28 binding agent can be used to provide a costimulatory signal. Illustrative examples of CD28 binding agents include but are not limited to: natural CD 28 ligands, e.g., a natural ligand for CD28 (e.g., a member of the B7 family of proteins, such as B7-1(CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In preferred embodiments, the T cells are activated with soluble anti-CD3 antibodies and stimulated to proliferate with anti-CD28 antibodies. In particular embodiments, the anti-CD3 antibodies and anti-CD8 antibodies are fixed, tethered, or bound to a bead, such as a paramagnetic bead, e.g., Dynabead.

In certain embodiments, the anti-CD3 antibodies and anti-CD8 antibodies are localized on the surface of a cell. In preferred embodiments, primary and costimulatory ligands, e.g., anti-CD3 antibodies and anti-CD28 antibodies are presented on an antigen presenting cell (e.g., an aAPC, dendritic cell, B cell, and the like) present in the PBMC fraction.

In particular embodiments, T cells are activated and stimulated for about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours. In particular embodiments, T cells are activated and stimulated for about 24 hours.

In particular embodiments, T cells are activated and stimulated for about 16 hours to about 30 hours, about 16 hours to about 24 hours, about 18 hours to about 24 hours, or about 20 hours to about 24 hours.

In preferred embodiments, the cells subjected to the activation and stimulation steps are transduced in the presence of a PI3K inhibitor, e.g., ZSTK474. Although the purpose of this step of the process is to transduce immune effector cells, other cells may be present and transduced, e.g., if PBMCs are used as the starting material then CD4⁺, CD8⁺, or CD4⁺ and CD8⁺ are transduced as well as other mononuclear cells such as monocytes, B cells, NK cells and NKT cells. In preferred embodiments, activated and stimulated T cells are transduced with a viral vector comprising a polynucleotide encoding a CAR. Illustrative examples of viral vector systems suitable for use in particular embodiments contemplated in particular embodiments include, but are not limited to, adeno-associated viral vectors (AAV), retroviral vectors e.g., lentiviral vectors, herpes simplex viral vectors, adenoviral vectors, and vaccinia viral vectors.

In preferred embodiments, cells are transduced with a lentivirus comprising a polynucleotide encoding a CAR. As used herein, the term “lentivirus” refers to a group (or genus) of complex retroviruses. Illustrative lentiviruses include, but are not limited to, HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (Hy); bovine immune deficiency virus (BIV); and simian immunodeficiency virus (SW). In one embodiment, HIV-1 based vector backbones (i.e., HIV cis-acting sequence elements) are preferred.

In various embodiments, a lentiviral vector contemplated herein comprises a chimeric 5′ long terminal repeat (LTR), e.g., chimeric CMV/5′ LTR promoter, and one or more, or all, of the following accessory elements: a cPPT/FLAP (Zennou, et al., 2000, Cell, 101:173), a Psi (4′) packaging signal (Clever et al., 1995. J. of Virology, Vol. 69, No. 4; pp. 2101-2109), an export element, e.g., RRE (Cullen et al., 1991. J. Virol. 65: 1053; and Cullen et al., 1991. Cell 58: 423), a poly (A) sequences, optionally a WPRE (Zufferey et al., 1999, J. Virol., 73:2886) or HPRE (Huang et al., Mol. Cell. Biol., 5:3864), an insulator element, a selectable marker, or a cell suicide gene, and a modified self-inactivating (SIN) 3′ LTR. “Self-inactivating” (SN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. In particular embodiments, a lentiviral vector is pseudotyped with vesicular stomatitis virus G-protein (VSV-G) envelope proteins to enable the vector to infect a broad range of cells. In certain embodiments, lentiviral vectors are produced according to known methods. See e.g., Kutner et at, BMC Biotechnol. 2009;9:10. doi: 10.1186/1472-6750-9-10; Kutner et al. Nat. Protoc. 2009;4(4):495-505. doi: 10.1038/nprot.2009.22.

In particular embodiments, after activation and stimulation, the cells are transduced for about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours. In particular embodiments, the cells are transduced for about 24 hours.

In particular embodiments, after activation and stimulation, the cells are transduced for about 16 hours to about 30 hours, about 16 hours to about 24 hours, about 18 hours to about 24 hours, or about 20 hours to about 24 hours.

In preferred embodiments, after transduction, cells are cultured in conditions that promote immune effector cell, e.g., T cells, CAR T cells, or anti-BCMA CAR T cells, proliferation or expansion in the presence of a PI3K inhibitor, e.g., ZSTK474. Unexpectedly, the inventors discovered that extremely shortened proliferation or expansion periods of 1, 2, 3, 4, 5, or 6 days (after transduction) produce a highly potent cell therapy product enriched in CD27⁺ cells, TCMs, and TSCMs.

In particular embodiments, conditions appropriate for T cell proliferation or expansion culture include culturing the cells in an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL- 10, IL- 12, IL-15, TGFβ, and TNF-α or any other additives suitable for the growth of cells known to the skilled artisan. Further illustrative examples of cell culture media include, but are not limited to RPMI 1640, Clicks, AIM-V, DMEM, MEM, a-MEM, F-12, X-Vivo 1 5, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Illustrative examples of other additives for T cell expansion include, but are not limited to, surfactant, plasmanate, pH buffers such as HEPES, and reducing agents such as N-acetyl-cysteine and 2-mercaptoethanol.

In preferred embodiments, T cells are cultured for proliferation or expansion for 1, 2, 3, 4, 5, or 6 days in T cell growth medium (TCGM) were prepared with X-VIVO 15 supplemented with 10 mM HEPES, 2 mM GlutaMax and 5% human AB serum. In preferred embodiments, the manufacturing process is carried out in the presence of one or more cytokines, preferably IL-2, IL-7, and/or IL-15, and more preferably, IL-2.

In particular embodiments, the cell proliferation or expansion phase is carried out for about 1 day to about 6 days, about 2 days to about 6 days, about 3 days to about 6 days, or about 4 days to about 6 days. In preferred embodiments, the cell proliferation or expansion phase is carried out for about 4 days to about 6 days.

In particular embodiments, the cell proliferation or expansion phase is carried out for about 1 day, about 2 days, about 3 days, about 4 days, about 5 days, or about 6 days. In preferred embodiments, the cell proliferation or expansion phase is carried out for about 4 days. In particular preferred embodiments, the cell proliferation or expansion phase is carried out for about 6 days.

In various embodiments, T cell compositions are manufactured in the presence of one or more inhibitors of the PI3K pathway. The inhibitors may target one or more activities in the pathway or a single activity. Without wishing to be bound to any particular theory, it is contemplated that treatment or contacting T cells with one or more inhibitors of the PI3K pathway during the stimulation, activation, and/or expansion phases of the manufacturing process preferentially increases young T cells, thereby producing superior therapeutic T cell compositions.

In various embodiments, a method of manufacturing CAR T cells comprises activating a population of T cells and stimulating the population of T cells to proliferate; transducing the T cells with a lentiviral vector comprising a polynucleotide encoding a CAR; and culturing the transduced T cells to proliferate for a period of about 4 days to about 6 days; wherein all of the method steps are performed in the presence of a PI3K inhibitor, and wherein the proliferated CAR T cells are enriched in TCM and TSCM cells compared to the manufacturing process wherein the transduced cells are cultured for a period of about 9 days in the PI3K inhibitor.

In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment in CD4+ T cells, having a TCM phenotype and an about 1.5 fold, about 2.0 fold, about 2.5 fold, about 3 fold, about 3.5 fold, about 4 fold, about 4.5 fold, or about 5 fold enrichment in CD8⁺ T cells, having a TSCM phenotype compared to the manufacturing process wherein the transduced cells are cultured for a period of about 9 days in the PI3K inhibitor.

In various embodiments, a method of manufacturing CAR T cells comprises activating a population of T cells and stimulating the population of T cells to proliferate; transducing the T cells with a lentiviral vector comprising a polynucleotide encoding a CAR, e.g., an anti-BCMA CAR that comprises the amino acid sequence set forth in SEQ ID NO: 1 (e.g., SEQ ID NO: 2); and culturing the transduced T cells to proliferate for a period of about 4 days to about 6 days; wherein all of the method steps are performed in the presence of a PI3K inhibitor, and wherein the proliferated CAR T cells are enriched in CD27⁺ cells compared to the manufacturing process wherein the transduced cells are cultured for a period of about 9 days in the PI3K inhibitor.

In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment in CD27⁺ T cells compared to the manufacturing process wherein the transduced cells are cultured for a period of about 9 days in the PI3K inhibitor.

In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment of, or increase in the number of, one or more CD27, CD25, CD127, TCF1, LEF1, CD28, and/or CCR7 expressing T cells compared to the manufacturing process wherein the transduced cells are cultured for a period of about 9 days in the PI3K inhibitor. In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment of, or increase in the number of, one or more CD27, CD25, CD127, TCF1, and/or LEF1 and/or CCR7 expressing T cells compared to the manufacturing process wherein the transduced cells are cultured for a period of about 9 days in the PI3K inhibitor. In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold decrease the number of T cells expressing one or more of Granzyme A, Granzyme B, Perform, T-bet, and EOMES compared to the manufacturing process wherein the transduced cells are cultured for a period of about 9 days in the PI3K inhibitor.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the proliferated T cells are enriched in CD27⁺CD4⁺ TCM and CD27⁺CD8⁺ TSCM cells compared to the manufacturing process wherein the transduced cells are cultured for a period of about 9 days in the PI3K inhibitor.

In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment in CD27⁺CD4⁺ T cells, having a TCM phenotype and an about 1.5 fold, about 2.0 fold, about 2.5 fold, about 3 fold, about 3.5 fold, about 4 fold, about 4.5 fold, or about 5 fold enrichment in CD27⁺CD8⁺ T cells, having a TSCM phenotype compared to the manufacturing process wherein the transduced cells are cultured for a period of about 9 days in the PI3K inhibitor.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the proliferated T cells are enriched in CD27⁺ and/or LEF1⁺ and/or CCR7⁺ and/or TCF1⁺ CD4⁺ TCM and CD27⁺ and/or LEF1⁺ and/or CCR7⁺ and/or TCF1⁺ CD8⁺ TSCM cells compared to the manufacturing process wherein the transduced cells are cultured for a period of about 9 days in the PI3K inhibitor.

In particular embodiments, a method of manufacturing anti-BCMA CAR T cells, comprising proliferation or expansion culture of about 4 days to about 6 days results in an about 1.5 fold, about 2.0 fold, about 2.5 fold, about 3 fold, about 3.5 fold, about 4 fold, about 4.5 fold, or about 5 fold enrichment in CD27⁺ and/or LEF¹+ and/or CCR7⁺ and/or TCF1⁺ CD4+ T cells, having a TCM phenotype and an about 1.5 fold, about 2.0 fold, about 2.5 fold, about 3 fold, about 3.5 fold, about 4 fold, about 4.5 fold, or about 5 fold enrichment in CD27⁺ and/or LEF1⁺ and/or CCR7⁺ and/or TCF1⁺ CD8⁺ T cells, having a TSCM phenotype compared to the manufacturing process wherein the transduced cells are cultured for a period of about 9 days in the PI3K inhibitor.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the gene expression signature of proliferated T cells have enriched or increased expression of one or more of, or all of, Nuclear Receptor Subfamily 4 Group A Member 2 (NR4A2), CD229 (LY9), Lin-7 Homolog A (LIN7A), Wingless-Type MMTV Integration Site Family, Member 5B (WNT5B), B cell CLL/lymphoma 6 (BCL6), Early Growth Response 1 (EGR1), Early Growth Response 2 (EGR2), Activating Transcription Factor 3 (ATF3), C-C motif chemokine 1 (CCL1), Interleukin 1A (IL-1A), and C-C motif chemokine 5 (CCL5) and have decreased expression of one or more of, or all of, NAD(P)H Quinone Dehydrogenase 1 (NQO1), Cyclin A1 (CCNA1), Interkleukin 17F (IL17F), Epithelial Membrane Protein 1 (EMP1), Small Nucleolar RNA Host Gene 19 (SNHG19), Proline Rich 22 (PRR 22), Immunoglobulin Like Domain Containing Receptor 2 (ILDR2), ATPase Family, AAA Domain Containing 3 (ATAD3), Naked Cuticle Homolog 2 (NKD2) and WD Repeat Domain 62 (WDR62).

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the gene expression signature of proliferated T cells have enriched or increased expression of CCL1, NR4A2, ATF3, CCL5, and WNT5B and have decreased expression of NKD2 and NQO1.

“Gene expression” refers to the relative levels of expression and/or pattern of expression of a gene in a biological sample, a population of T cells, e.g., anti-BCMA CAR T cells, manufactured in the presence or absence of a PI3K inhibitor, or manufactured for different lengths of time in the presence of a PI3K inhibitor. Gene expression may be measured at the level of cDNA, RNA, mRNA, or combinations thereof. Methods to measure gene expression include but are not limited to quantitative real-time, PCR, high-density oligonucleotide arrays, Nanostring transcriptome profiling, or RNA sequencing (RNA-Seq).

In particular embodiments, T cells including CAR T cells, e.g., anti-BCMA CAR T cells, manufactured using a seven day manufacturing process using a PI3K inhibitor contemplated herein are characterized by at least a 1.5-fold or at least a 2-fold increase in expression of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B compared to T cells manufactured using a 10 day manufacturing process contemplated herein. T cells manufactured using the seven day process using a PI3K inhibitor are further characterized by a unique gene expression signature wherein expression of 1, 2, 3, 4, 5, 6, 7, 8, 9, 10, or all 11 of the signature genes selected from the group consisting of: NR4A2, LY9, LIN7A, WNT5B, BCL6, EGR1, EGR2, ATF3, CCL1, IL-1A, and CCL5 is increased at least 1.5-fold or at least 2-fold, compared to T cells manufactured using the 10 day process using the PI3K inhibitor.

In particular embodiments, T cells including CAR T cells, e.g., anti-BCMA CAR T cells, manufactured using a seven day manufacturing process using a PI3K inhibitor contemplated herein are characterized by at least a 1.5-fold or at least a 2-fold decrease in expression of (i) NQ01, CCNA1, IL17F, EMP1, SNHG19, PRR 22, ILDR2, ATAD3, NKD2 and WDR62 or (ii) NKD2 and NQO1 compared to T cells manufactured using a 10 day manufacturing process contemplated herein. T cells manufactured using the seven day process using a PI3K inhibitor are further characterized by a unique gene expression signature wherein expression of 1, 2, 3, 4, 5, 6, 7, 8, 9, or all 10 of the signature genes selected from the group consisting of: NQO1, CCNA1, IL17F, EMP1, SNHG19, PRR 22, ILDR2, ATAD3, NKD2 and WDR62 is decreased at least 1.5-fold or at least 2-fold, compared to T cells manufactured using the 10 day process using the PI3K inhibitor.

In particular embodiments, T cells including CAR T cells, e.g., anti-BCMA CAR T cells, manufactured using a seven day manufacturing process using a PI3K inhibitor contemplated herein are characterized by at least a 1.5-fold or at least a 2-fold increase in expression of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B; and at least a 1.5-fold or at least a 2-fold decrease in expression of (i) NQO1, CCNA1, IL17F, EMP1, SNHG19, PRR 22, ILDR2, ATAD3, NKD2 and WDR62 or (ii) NKD2 and NQO1 compared to T cells manufactured using a 10 day manufacturing process contemplated herein. T cells manufactured using the seven day process using a PI3K inhibitor are further characterized by a unique gene expression signature wherein expression of 1, 2, 3, 4, 5, 6, 7, 8, 9, 10, or all 11 of the signature genes selected from the group consisting of: NR4A2, LY9, LIN7A, WNT5B, BCL6, EGR1, EGR2, ATF3, CCL1, IL-1A, and CCL5 is increased at least 1.5-fold or at least 2-fold and expression of 1, 2, 3, 4, 5, 6, 7, 8, 9, or all 10 of the signature genes selected from the group consisting of NQO1, CCNA1, IL17F, EMP1, SNHG19, PRR 22, ILDR2, ATAD3, NKD2 and WDR62 is decreased at least 1.5-fold or at least 2-fold, compared to T cells manufactured using the 10 day process using the PI3K inhibitor.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the proliferated T cells are enriched in TCM and TSCM cells and wherein the gene expression of 1, 2, 3, 4, 5, 6, 7, 8, 9, 10, or all of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNTSB is at least 1.5-fold greater in the cultured T cells cultured to proliferate for a period of about 4 days to about 6 days compared to T cells cultured to proliferate for a period of about 9 days.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the proliferated T cells are enriched in TCM, TSCM cells and wherein the gene expression of 1, 2, 3, 4, 5, 6, 7, 8, 9, or all of (i) NQ01, CCNA1, IL17F, EMP1, SNHG19, PRR 22, ILDR2, ATAD3, NKD2 and WDR62 or (ii) NKD2 and NQO1 is at least 1.5-fold less in the cultured T cells cultured to proliferate for a period of about 4 days to about 6 days compared to T cells cultured to proliferate for a period of about 9 days.

In various embodiments, a method of manufacturing anti-BCMA CAR T cells comprises activating a population of T cells and stimulating the population of T cells to proliferate; transducing the T cells with a lentiviral vector comprising a polynucleotide encoding an anti-BCMA CAR that comprises the amino acid sequence set forth in SEQ ID NO: 1 (e.g., SE QID NO: 2); and culturing the transduced T cells to proliferate for a period of about 4 days to about 6 days; wherein all of the method steps are performed in the presence of a PI3K inhibitor, and wherein the proliferated T cells are enriched in TCM, TSCM cells and wherein the gene expression of 1, 2, 3, 4, 5, 6, 7, 8, 9, 10, or all of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNTSB is at least 1.5-fold greater and the gene expression of 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQOlis at least 1.5-fold less, in the cultured T cells cultured to proliferate for a period of about 4 days to about 6 days compared to T cells cultured to proliferate for a period of about 9 days.

Manufacturing methods contemplated herein may further comprise cryopreservation of PBMCs prior to initiation of the manufacturing process and/or cryopreservation of the manufactured T cell composition. Cryopreservation of adoptive cell therapies allows for storage, testing, transportation, and release of the therapeutic for use in a human subject. T cells are cryopreserved such that the cells remain viable upon thawing. When needed, the cryopreserved cells can be thawed, grown and expanded for more such cells. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. NY. Acad. Sci., 1960; 85: 576), polyethylene glycol (Sloviter and Ravdin, Nature, 1962; 196: 48), and CryoStor CS10, CryoStor CSS, and CryoStor CS2. In preferred embodiments, the manufactured T cells are formulated in a solution comprising 50:50 PlasmaLyte A to CryoStor CS10. The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

### D. Chimeric Antigen Receptors

The methods contemplated herein are used to manufacture more potent adoptive cell therapies that redirect cytotoxicity of immune effector cells toward cancer cells expressing a target antigen. In preferred embodiments, manufacturing methods contemplated herein comprise transducing activated and stimulated T cells with a viral vector encoding a chimeric antigen receptor (CAR) to redirect the immune effector cells.

CARs are molecules that combine antibody-based specificity for a target antigen (e.g., tumor antigen) with a T cell receptor-activating intracellular domain to generate a chimeric protein that exhibits a specific anti-tumor cellular immune activity. The CARs contemplated herein comprise a signal peptide, an extracellular domain that binds to a specific target antigen (also referred to as a binding domain or antigen-specific binding domain), a transmembrane domain and one or more intracellular signaling domains.

In particular embodiments, CARs comprise an extracellular binding domain that specifically binds to a target polypeptide. In particular embodiments, the extracellular binding domain comprises an antibody or antigen binding fragment thereof. In one preferred embodiment, the binding domain comprises an scFv. In another preferred embodiment, the binding domain comprises one or more camelid VHH antibodies or a single domain antibody (sdAb).

In particular embodiments, a CAR comprises an extracellular domain that binds an antigen selected from the group consisting of: alpha folate receptor (FRa), avf36 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRLS), fetal acetylcholinesterase receptor (AchR), ganglioside G2 (GD2), ganglioside G3 (GD3), Glypican-3 (GPC3), EGFR family including ErbB2 (HER2), IL-10Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBPS, ULBP6, vascular endothelial growth factor receptor 2 (VEGFR2), and Wilms tumor 1 (WT-1).

In preferred embodiments, the CAR comprises an extracellular domain that binds B cell maturation antigen.

In a particular embodiment, a CAR comprises a hinge domain. Illustrative hinge domains include but are not limited to the hinge region derived from the extracellular regions of type 1 membrane proteins such as CD8α, and CD4, which may be wild-type hinge regions from these molecules or may be altered. In a preferred embodiment, a CAR comprises a CD8α hinge region.

The transmembrane (TM) domain of the CAR fuses the extracellular binding portion and intracellular signaling domain and anchors the CAR to the plasma membrane of the immune effector cell. The TM domain may be derived either from a natural, synthetic, semi-synthetic, or recombinant source. Illustrative TM domains may be derived from (i.e., comprise at least the transmembrane region(s) of the alpha, beta, gamma, or delta chain of a T-cell receptor, CDR, CD3, CD4, CDS, CD8a, CD9, CD 16, CD22, CD27, CD28, CD33, CD37, CD45, CD64, CD71, CD80, CD86, CD 134, CD137, CD152, CD 154, AMN, and PDCD1.

In a preferred embodiment, a CAR comprises a TM domain derived from CD8α. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine linker provides a particularly suitable linker.

In preferred embodiments, a CAR comprises an intracellular signaling domain that comprises one or more costimulatory signaling domains and a primary signaling domain.

Primary signaling domains that act in a stimulatory manner may contain signaling motifs which are known as immunoreceptor tyrosine-based activation motifs or ITAMs.

Illustrative examples of ITAM containing primary signaling domains suitable for use in CARs contemplated in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more costimulatory signaling domains. The intracellular primary signaling and costimulatory signaling domains may be linked in any order in tandem to the carboxyl terminus of the transmembrane domain.

In particular embodiments, a CAR comprises one or more costimulatory signaling domains to enhance the efficacy and expansion of T cells expressing CAR receptors.

Illustrative examples of such costimulatory molecules suitable for use in CARs contemplated in particular embodiments include, but are not limited to, TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD94, CD134 (0X40), CD137 (4-1BB), CD278 (ICOS), DAP10, LAT, SLP76, TRAT1, TNFR2, and ZAP70. In one embodiment, a CAR comprises one or more costimulatory signaling domains selected from the group consisting of CD28, CD137, and CD134, and a CD3 primary signaling domain.

In preferred embodiments, a CAR comprises a CD8a signal peptide; an extracellular domain that binds BCMA; a CD8a hinge and transmembrane domain; a CD137 costimulatory domain, and a CD137; and a CD3 primary signaling domain. In a more preferred embodiment, the anti-BCMA CAR comprises the amino acid sequence set forth in SEQ ID NO: 1, and an even more preferred embodiment, the anti-BCMA CAR comprises the polynucleotide sequence set forth in SEQ ID NO: 2.

### E. Compositions and Formulations

The compositions contemplated herein comprise a therapeutically effective amount of CAR T cells. In preferred embodiments, compositions contemplated herein comprises a therapeutically effective amount of anti-BCMA CAR T cells. Compositions include but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

In preferred embodiments, compositions contemplated herein comprise a cGMP manufactured population of CAR T cells enriched in T cells expressing one or more of CD27, LEF1, and TCF1 on the cell surface. In preferred embodiments, an enriched population of CAR T cells manufactured using a 5 day to 7 day process in the presence of a PI3K inhibitor comprise at least 10% CD27⁺, at least 15% CD27⁺, at least 20% CD27⁺, at least 25% CD27⁺, at least 30% CD27⁺, at least 35% CD27⁺, at least 40% CD27⁺, at least 45% CD27⁺, or at least 50% CD27⁺ CAR T cells. In particular embodiments, an enriched population of CAR T cells manufactured using a 5 day to 7 day process in the presence of a PI3K inhibitor comprise at least 10% CD27⁺, LEF1⁺, and/or TCF1⁺, at least 15% CD27⁺, LEF1⁺, and/or TCF1⁺, at least 20% CD27⁺, LEF1⁺, and/or TCF1⁺, at least 25% CD27⁺, LEF1⁺, and/or TCF1⁺, at least 30% CD27⁺, LEF1⁺, and/or TCF1⁺, at least 35% CD27⁺, LEF1⁺, and/or TCF1⁺, at least 40% CD27⁺, LEF1⁺, and/or TCF1⁺, at least 45% CD27⁺, LEF1⁺, and/or TCF1⁺, or at least 50% CD27⁺, LEF1⁺, and/or TCF1 CAR T cells. In particular embodiments, an enriched population of CAR T cells manufactured using a 5 day to 7 day process in the presence of a PI3K inhibitor comprise at least 10% CD27⁺LEF1⁺, at least 15% CD27⁺LEF1⁺, at least 20% CD27+LEF1+TCF1+, at least 25% CD27LEF1⁺, at least 30% CD27⁺LEF1⁺, at least 35% CD27⁺LEF1⁺, at least 40% CD27⁺LEF1⁺TCF1⁺, at least 45% CD27⁺LEF1⁺, or at least 50% CD27⁺LEF1⁺TCF1⁺ CAR T cells. In particular embodiments the T cells are also CCR7⁺.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch: cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount, and more preferably a therapeutically effective amount, of CAR-expressing T cells contemplated herein.

As used herein, the term “amount” or “dose” refers to “an amount effective,” “a dose effective,” “an effective amount,” or “an effective dose” of a CAR T cell sufficient to achieve a beneficial or desired prophylactic or therapeutic result, including clinical results.

A “therapeutically effective amount” or “therapeutically effective dose” of a CAR T cell is also one in which any toxic or detrimental effects of a CAR T cell, e.g., CRS, are outweighed by the therapeutically beneficial effects. The term “therapeutically effective amount” includes an amount that is effective to “treat” a subject (e.g., a patient). In one embodiment, the therapeutically effective dose is the minimal effective dose (MED) of CAR T cells to treat multiple myeloma in a subject. In one embodiment, the therapeutically effective dose is the maximum tolerated dose (MTD) of anti-BCMA CAR T cells that does not lead to unresolvable CRS in a subject. In preferred embodiments, a therapeutically effective amount of CAR T cells, e.g., anti-BCMA CAR T cells manufactured using a 5 day or 7 day manufacturing process in the presence of a PI3K inhibitor, is administered to a subject, wherein the amount of cells is less than the amount of cells necessary to achieve a comparable outcome from CAR T cells manufactured using a 10 day manufacturing process using a PI3K inhibitor.

In particular embodiments, compositions are preferably formulated for parenteral administration, e.g., intravascular (intravenous or intraarterial), administration. In a preferred embodiment, the compositions contemplated herein are intravenously infused into the subject in a single dose.

In one embodiment, the amount of CAR^{T} T cells in a composition administered to a subject is at least about 5.0×10⁷ cells, at least about 15.0×10⁷ cells, at least about 45.0×10⁷ cells, at least about 80.0×10⁷ cells, or at least about 12.0×10⁸ cells.

In one embodiment, the amount of CAR^{T} T cells in a composition administered to a subject is greater than about 5.0×10⁷ cells, greater than about 15.0×10⁷ cells, greater than about 45.0×10⁷ cells, greater than about 80.0×10⁷ cells, or greater than about 12.0×10⁸ cells.

In one embodiment, the amount of CAR^{T} T cells in a composition administered to a subject is between about 5.0×10⁷ cells to about 15.0×10⁷ cells, between about 5.0×10⁷ cells to about 45.0×10⁷ cells, between about 5.0×10⁷ cells to about 80.0×10⁷ cells, or between about 5.0×10⁷ cells to about 12.0×10⁸ cells.

For uses provided herein, the cells are generally in a volume of a liter or less, can be 500 mLs or less, even 250 mLs or 100 mLs or less.

In particular embodiments, pharmaceutical compositions comprise a therapeutically effective amount of CAR T cells, in combination with one or more pharmaceutically or physiologically acceptable carriers, diluents or excipients.

Pharmaceutical compositions comprising a therapeutically effective dose of CAR T cells may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In particular embodiments, CAR T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising CAR T cells contemplated herein are formulated in a solution comprising PlasmaLyte A.

In another preferred embodiment, compositions comprising CAR T cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising CAR T cells contemplated herein are formulated in a solution comprising 50:50 PlasmaLyte A to CryoStor CS10.

### F. Therapeutic Methods

The modified T cells manufactured by the methods contemplated herein provide improved adoptive immunotherapy for use in the treatment of various conditions including without limitation, cancer, infectious disease, autoimmune disease, inflammatory disease, and immunodeficiency. In particular embodiments, the specificity of a primary T cell is redirected to tumor or cancer cells by genetically modifying the primary T cell with a CAR contemplated herein.

In particular embodiments, CAR T cell compositions manufactured with the methods contemplated herein are used in the treatment of solid tumors or cancers including, without limitation, liver cancer, pancreatic cancer, lung cancer, breast cancer, bladder cancer, brain cancer, bone cancer, thyroid cancer, kidney cancer, or skin cancer.

In particular embodiments, CAR T cell compositions manufactured with the methods contemplated herein are used in the treatment of liquid tumors, including but a leukemia, including acute leukemia (e.g., ALL, AML, and myeloblastic, promyelocytic, myelomonocytic, monocytic and erythroleukemia), chronic leukemias (e.g., CLL, SLL, CML, HCL), polycythemia vera, lymphoma, Hodgkin's disease, non-Hodgkin's lymphoma, multiple myeloma, Waldenstrom's macroglobulinemia, and heavy chain disease.

In particular embodiments, CAR T cell compositions manufactured with the methods contemplated herein are used in the treatment of B-cell malignancies, including but not limited to multiple myeloma (MM), non-Hodgkin's lymphoma (NHL), and chronic lymphocytic leukemia (CLL).

Multiple myeloma is a B-cell malignancy of mature plasma cell morphology characterized by the neoplastic transformation of a single clone of these types of cells. These plasma cells proliferate in BM and may invade adjacent bone and sometimes the blood. Variant forms of multiple myeloma include overt multiple myeloma, smoldering multiple myeloma, plasma cell leukemia, non-secretory myeloma, IgD myeloma, osteosclerotic myeloma, solitary plasmacytoma of bone, and extramedullary plasmacytoma (see, for example, Braunwald, et al. (eds), Harrison's Principles of Internal Medicine, 15th Edition (McGraw-Hill 2001)).

Non-Hodgkin lymphoma encompasses a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. For example, non-Hodgkin's lymphoma can be divided into aggressive (fast-growing) and indolent (slow-growing) types. Although non-Hodgkin lymphomas can be derived from B-cells and T-cells, as used herein, the term “non-Hodgkin lymphoma” and “B-cell non-Hodgkin lymphoma” are used interchangeably. B-cell non-Hodgkin lymphomas (NHL) include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. Lymphomas that occur after bone marrow or stem cell transplantation are usually B-cell non-Hodgkin lymphomas.

Chronic lymphocytic leukemia (CLL) is an indolent (slow-growing) cancer that causes a slow increase in immature white blood cells called B lymphocytes, or B cells. Cancer cells spread through the blood and bone marrow, and can also affect the lymph nodes or other organs such as the liver and spleen. CLL eventually causes the bone marrow to fail. Sometimes, in later stages of the disease, the disease is called small lymphocytic lymphoma.

In particular embodiments, compositions comprising a therapeutically effective amount of anti-BCMA CAR T cells are administered to a subject to treat multiple myeloma or lymphoma.

In particular embodiments, compositions comprising a therapeutically effective amount of anti-BCMA CAR T cells are administered to a subject to treat relapsed/refractory multiple myeloma. “Relapse” refers to the diagnosis of return, or signs and symptoms of return, of a cancer after a period of improvement or remission. “Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

In particular embodiments, compositions contemplated herein are administered to a subject with relapsed/refractory multiple myeloma that has been unsuccessfully treated with one, two, three or more treatments, including at least one proteasome inhibitor and/or an immunomodulatory drug (IMiD). In one embodiment, the subject's multiple myeloma is refractory to three treatment regimens, including at least one proteasome inhibitor and an IMiD. In one embodiment, the subject's multiple myeloma is double-refractory to one or more treatment regimens.

Illustrative examples of proteasome inhibitors to which subject's multiple myeloma is refractory include, but are not limited to, bortezomib, and carfilzomib.

Illustrative examples of IMiDs to which subject's multiple myeloma is refractory include, but are not limited to thalidomide, lenalidomide, and pomalidomide.

Illustrative examples of other treatments, to which multiple myeloma may be refractory include, but are not limited to, dexamethasone, and antibody-based therapies selected from the group consisting of elotuzumab, daratumumab, MOR03087, isatuximab, bevacizumab, cetuximab, siltuximab, tocilizumab, elsilimomab, azintrel, rituximab, tositumomab, milatuzumab, lucatumumab, dacetuzumab, figitumumab, dalotuzumab, AVE1642, tabalumab, pembrolizumab, pidilizumab, and nivolumab.

In one embodiment, the subject's multiple myeloma is refractory to treatment with daratumumab.

In particular embodiments, the subject's multiple myeloma is refractory to treatment with an IMiD, a proteasome inhibitor, and dexamethasone.

Methods contemplated herein, may further comprise treating a subject with relapsed/refractory multiple myeloma with an autologous hematopoietic stem cell transplant, prior to the administration of the anti-BCMA CAR T cell composition.

Methods contemplated herein, may further comprise lymphodepleting the subject prior to administration of an anti-BCMA CAR T cell composition contemplated herein, e.g., for example, the lymphodepleting chemotherapy ends 1-4 days (e.g., 1, 2, 3, or 4 days) prior to the administration. In particular embodiments, the lymphodepletion comprises administering one or more of melphalan, cytoxan, cyclophosphamide, and fludarabine. In one embodiment the subject is lymphodepleted with cyclophosphamide 300 mg/m2 and fludarabine 30 mg/m2 prior to administration of an anti-BCMA CAR T cell composition contemplated herein.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Improved Manufacturing Processes

Cells were harvested multiple myeloma donors by leukapheresis and PBMCs were isolated using density gradient on a Cell Saver Elite. PBMCs were washed and then resuspended in T cell growth medium (TCGM) with 2501U IU/mL IL-2. Pre- and post-wash cell counts, viability, and PBMC FACS analysis were performed. Washed PBMCs were cryopreserved until activation or used fresh. On day 0, T cells were activated and stimulated by culturing the PBMCs in TCGM with 250 IU/mL IL-2, 1μM ZSTK474 (CAS NO. 475110-96-4), 50 ng/mL of anti-CD3 antibody, and 50 ng/mL of anti-CD28 antibody to the culture and cultured for about 18-24 hours. The PBMC culture was transduced with a lentivirus encoding an anti-BCMA CAR (e.g., SEQ ID NO: 1, SEQ ID NO: 2) for about 18 to about 24 hours. The PBMC culture was then cultured for T cell expansion in TCGM containing 250 IU/mL of IL-2 and 1 μM ZSTK474 for 4 days, 6 days, or 9 days (5 day, 7 day, 10 day manufacturing processes, respectively). At each of the one or more days of expansion, aliquots of the cells were optionally taken and cells were counted, viability determined, cryopreserved, and characterized for PBMCs using FACS analysis. Expanded cells were recovered and washed and cryopreserved in a controlled rate freezer at a temperature of at least -80° C. and stored in the vapor phase of a liquid nitrogen storage tank.

### Example 2

### Improved Manufacturing Processes Modulate T Cell Phenotype

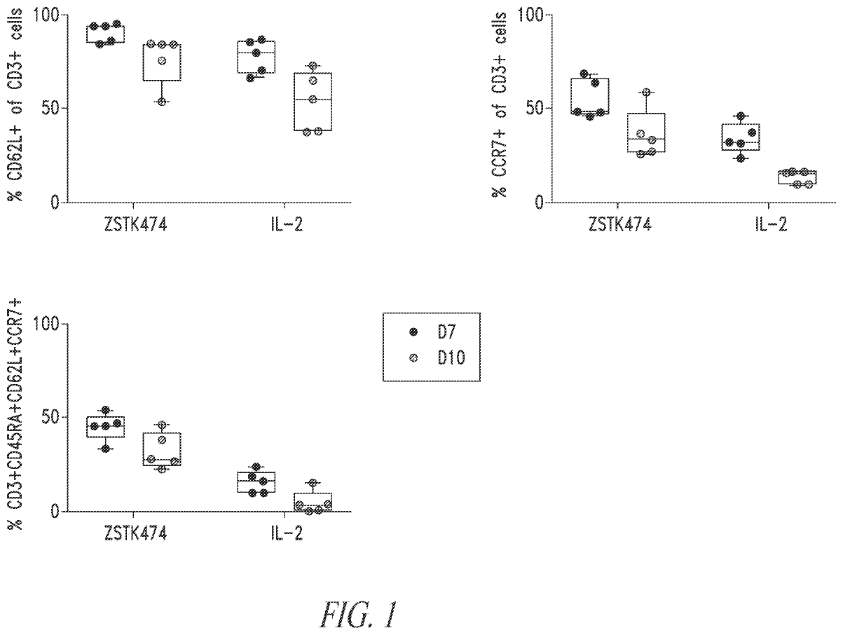

Five multiple myeloma donor PBMC cell lots were used to manufacture anti-BMCA CAR T cells using a 7 day or 10 day manufacturing process described in Example 1 in the presence or absence of the PI3K inhibitor ZSTK474. At the end of the T cell expansion culture, cells were stained with anti-human antibodies against CD3, CD62L, CCR7, and CD45RA and analyzed by flow cytometry. Each dot plot was gated on viable CD3⁺ lymphocytes. Anti-BCMA CAR T cell drug products (DP) manufactured in the presence of ZSTK474 for 7 days have increased marker expression for more potent T cell phenotypes compared to anti-BCMA CAR T cell DPs manufactured in the presence of ZSTK474 for 10 days or manufactured in the absence of the PI3K inhibitor. FIG. 1.

### Example 3

### Improved Manufacturing Processes Modulate T Cell Differentiation

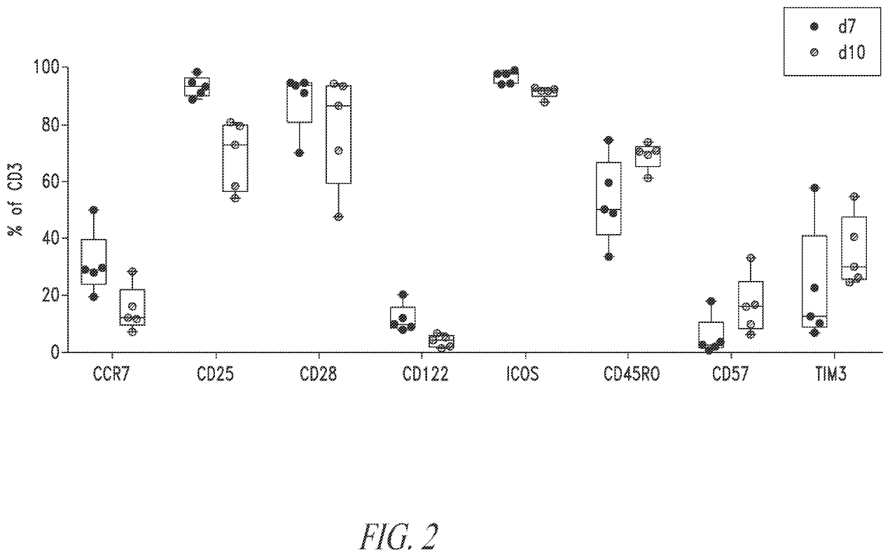

Five multiple myeloma donor PBMC cell lots were used to manufacture anti-BMCA CAR T cells using a 7 day or 10 day manufacturing process described in Example 1 in the presence of the PI3K inhibitor ZSTK474. At the end of the T cell expansion culture, cells were stained with metal labeled anti-human antibodies against CCR7, CD25, CD28, CD122, ICOS, CD45RO, CD57, and TIM3 and analyzed by CyTOF. Each dot plot was gated on viable CD3⁺ lymphocytes. Anti-BCMA CAR T cell DP manufactured in the presence of ZSTK474 for 7 days have increased marker expression for less differentiated T cell phenotypes and decreased marker expression for more differentiated T cell phenotypes compared to anti-BCMA CAR T cell DPs manufactured in the presence of ZSTK474 for 10 days. FIG. 2.

### Example 4

### Improved Manufacturing Processes Enrich CD27⁺T Cells

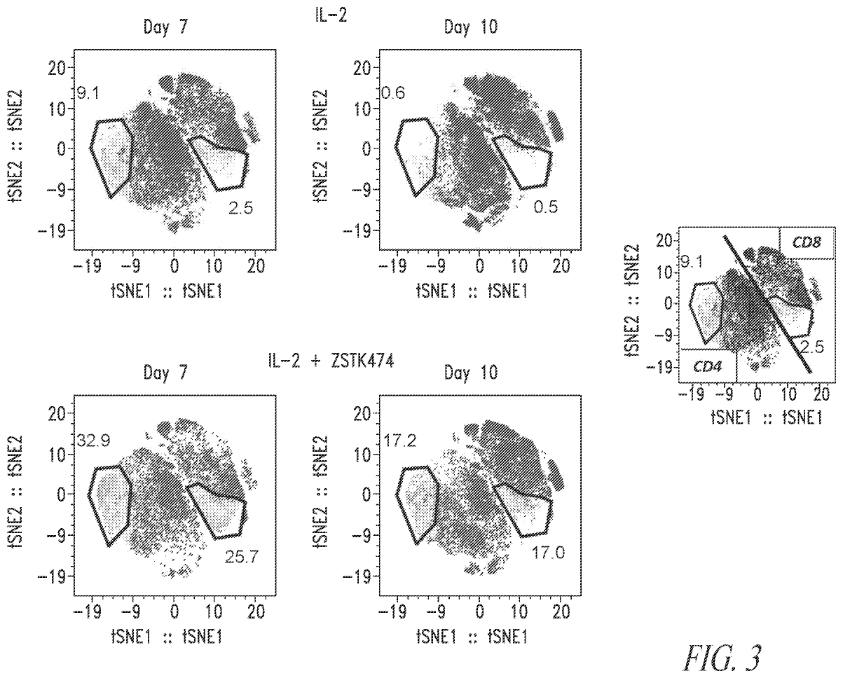

Five multiple myeloma donor PBMC cell lots were used to manufacture anti-BMCA CAR T cells using a 7 day or 10 day manufacturing process described in Example 1 in the presence or absence of the PI3K inhibitor ZSTK474. At the end of the T cell expansion culture, cells were stained with metal labeled anti-human antibodies against CD4, CD8, and CD27 and analyzed by CyTOF. VISNE plots show CD27 expression in different cell populations. Gated populations represent CD27⁺ enriched T cells. Anti-BCMA CAR T cell DP manufactured in the presence of ZSTK474 for 7 days have unexpected and dramatic increases in CD27⁺, LEF¹⁺, and/or TCF1⁺ enriched T cells compared to anti-BCMA CAR T cell DPs manufactured in the presence of ZSTK474 for 10 days or in the absence of the PI3K inhibitor. FIG. 3.

### Example 5

### Enriched CD27⁺T Cell Populations Have Potent T Cell Phenotype

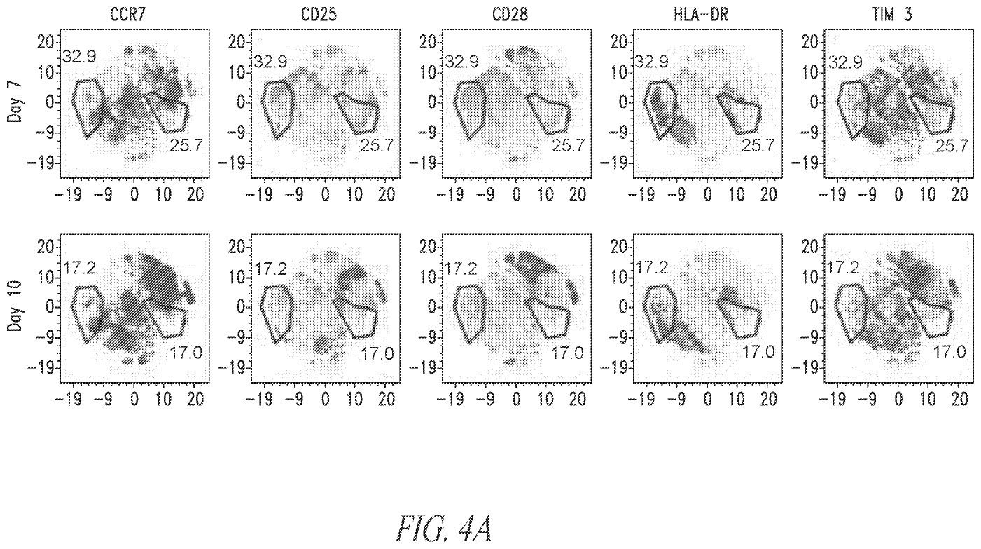

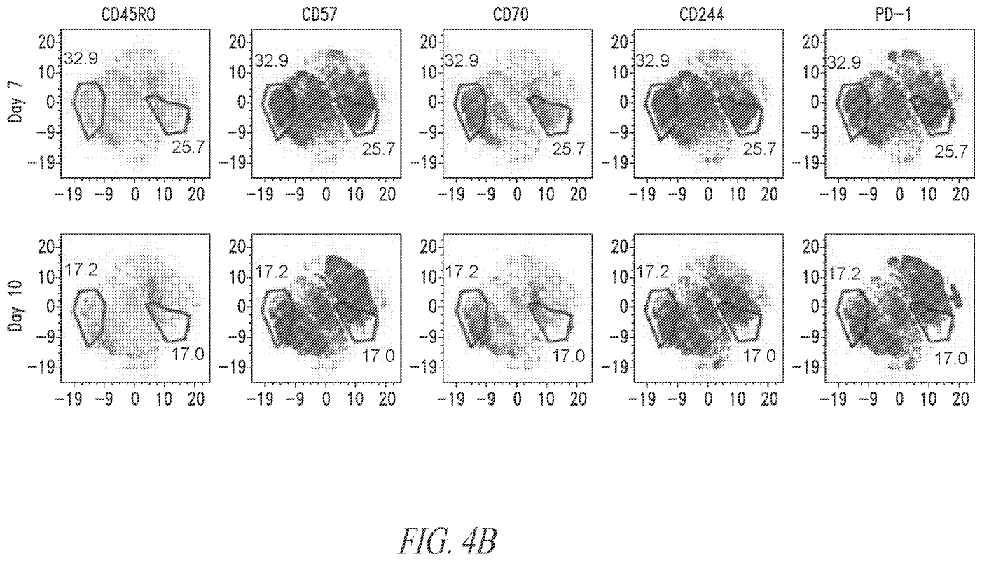

Five multiple myeloma donor PBMC cell lots were used to manufacture anti-BMCA CAR T cells using a 7 day or 10 day manufacturing process described in Example 1 in the presence of the PI3K inhibitor ZSTK474. At the end of the T cell expansion culture, cells were stained with metal labeled anti-human antibodies against CCR7, CD25, CD28, HLA-DR, and TIM3 (FIG. 4A) and CD45RO, CD57, CD70, CD244, and PD-1 (FIG. 4B) and analyzed by CyTOF. VISNE plots show marker expression in different cell populations. Gated populations represent CD27⁺ enriched T cells. Anti-BCMA CAR T cell DP manufactured in the presence of ZSTK474 for 7 days have increased marker expression for less differentiated T cell phenotypes and decreased marker expression for more differentiated T cell phenotypes compared to anti-BCMA CAR T cell DPs manufactured in the presence of ZSTK474 for 10 days. FIGS. 4A-4B.

### Example 6

### Improved Manufacturing Processes Modulate CD27⁺T Cell Activation Profile

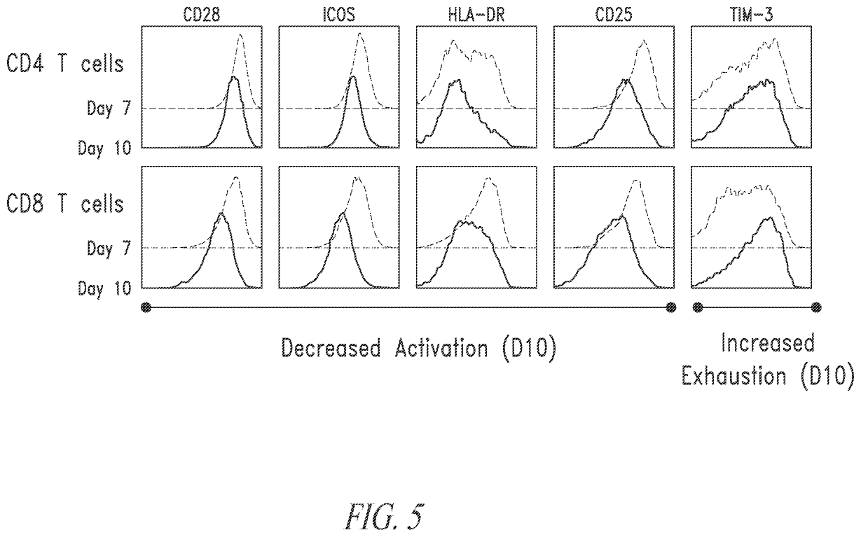

Five multiple myeloma donor PBMC cell lots were used to manufacture anti-BMCA CAR T cells using a 7 day or 10 day manufacturing process described in Example 1 in the presence of the PI3K inhibitor ZSTK474. At the end of the T cell expansion culture, cells were stained with metal labeled anti-human antibodies against CD27, CD28, ICOS, HLA-DR, CD25, and TIM3 and analyzed by CyTOF. T cell phenotypes of CD27⁺ enriched cells identified by VISNE were analysis for marker expression in CD4⁺ T cells (FIG. 5, top) and CD8⁺ T cells (FIG. 5, bottom). Anti-BCMA CAR T cell DP manufactured in the presence of ZSTK474 for 10 days have a decreased activation profile and increased exhaustion profile compared to anti-BCMA CAR T cell DPs manufactured in the presence of ZSTK474 for 7 days.

### Example 7

### Improved Manufacturing Processes Modulate T Cell Gene Expression

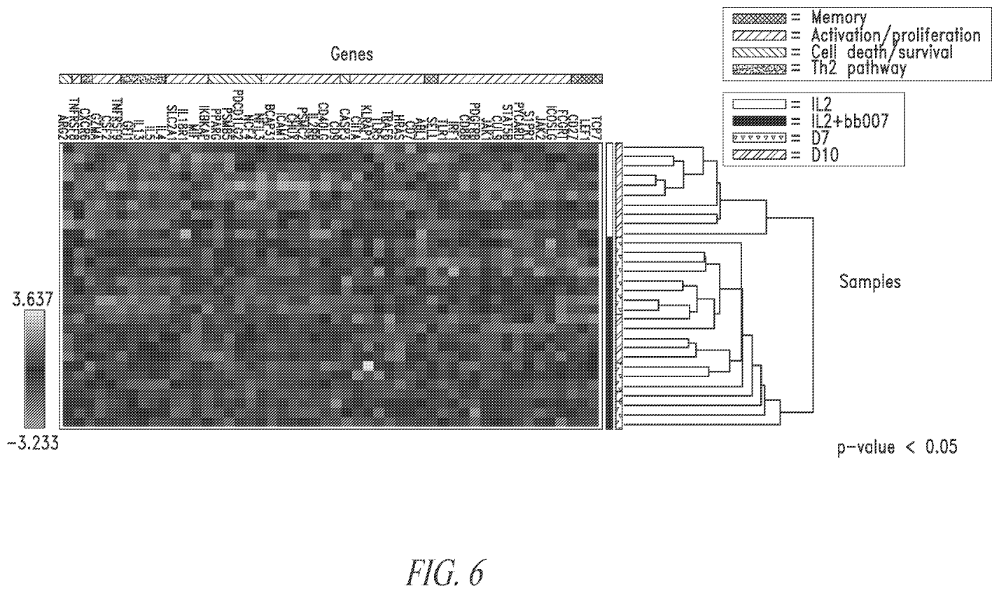

Multiple myeloma PBMC lots were used to manufacture anti-BCMA CAR T cells as described in Example 1 in the absence of the PI3K inhibitor ZSTK474 for 7 days (n=1) or 10 days (n=13) or in the presence of the PI3K inhibitor for 7 days (n=10) or 10 days (n=6). About 10Ong of total RNA was extracted from anti-BCMA CAR T cell DPs and mixed with the Immunology V2 probe kit from Nanostring and the transcriptional profile analyzed. A heatmap of the top 50 differentially expressed genes between manufacturing conditions is shown in FIG. 6. Anti-BCMA CAR T cell DPs manufactured for 7 days generally show increased expression of T cell memory phenotype genes and genes associated with T cell activation and proliferation and decreased expressed of genes associated with cell death compared to DPs manufactured for 10 days.

### Example 8

### Anti-BCMA CAR T Cells In A Daudi Tumor Mouse Model

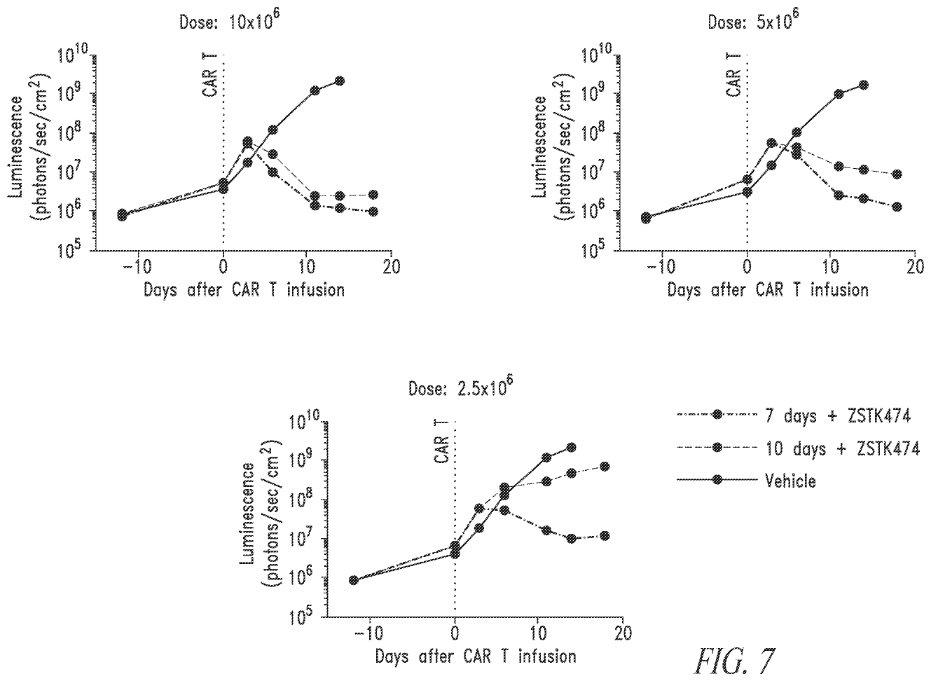

A Daudi tumor mouse model was established to compare the efficacy among the drug products manufactured with the 7 day and 10 day processes. Healthy donor PBMCs were activated and stimulated, transduced with a lentiviral vector encoding an anti-BCMA CAR, and expanded in the presence of IL-2 and PI3K inhibitor for 7 days or 10 days (see Example 1). NSG mice were injected intravenously with 2×10⁶ firefly luciferase labeled Daudi tumor cells 10 days before adoptive cell therapy. Mice were injected with 2.5, 5 or 10×10⁶ anti-BCMA CAR^{T} T cells or T cells transduced with vehicle. The tumor burden was monitored by luminescence. Anti-BCMA CAR T cells manufactured with the 7 day process show better efficacy, evidenced by increased ability to control tumor growth at lower CAR^{T} doses, than cells manufactured at 10 days. FIG. 7.

### Example 9

### Anti-BCMA CAR T Cell Phenotypes

Fifteen multiple myeloma donor PBMC cell lots were used to manufacture anti-BMCA CAR T cells using a 7 day or 10 day manufacturing process described in Example 1 in the presence of the PI3K inhibitor ZSTK474. At the end of the T cell expansion culture, cells were stained with a panel of ˜36 T cell phenotyping metal labeled anti-human antibodies and analyzed with CyTOF. The phenotyping antibodies enable discrimination among the following T cell phenotypes: Naive T cells (Tnaive), Central memory T cells (TCM), Effector memory T cells (EM), Effector T cells (TEff), and Stem cell memory T cells (TSCM). The T stem cell memory subset is identified by CD95 expression in the Naive T cell quadrant (CCR7⁺CD45RO⁻). The data presented shows each DP lot analyzed as a function of the % of CD27⁺ enriched cells vs. T cell subset. CD27⁺CD4⁺ T cells, positively correlate with a TCM like phenotype, whereas CD27⁺CD8⁺ T cells positively correlate with a TSCM like phenotype. FIG. 8.

### Example 10

### CD8⁺ Anti-BCMA CAR T Cell Phenotypes

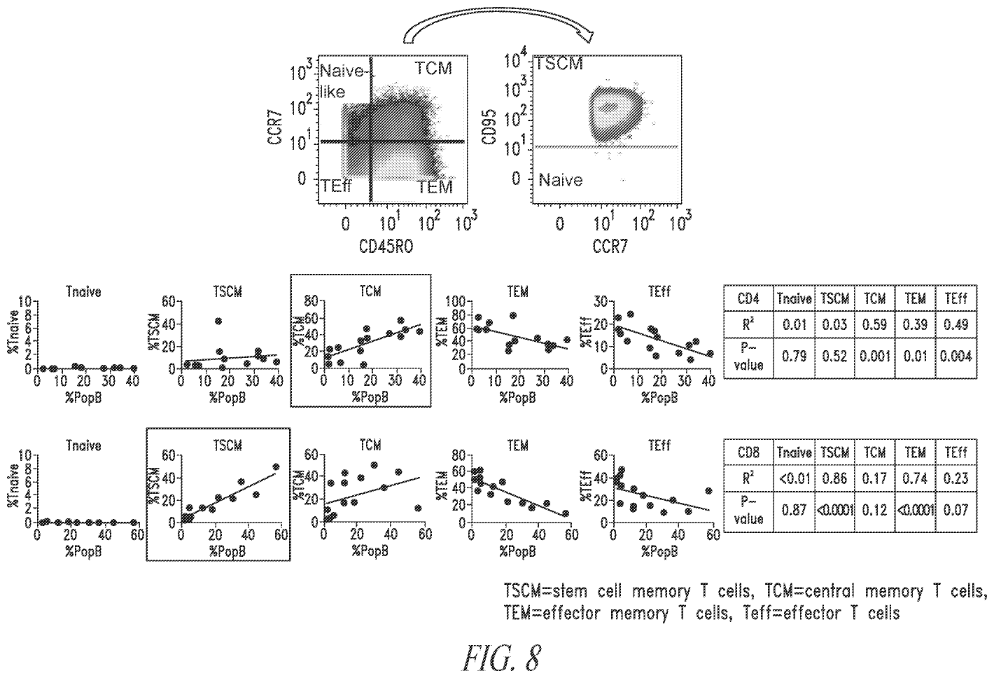

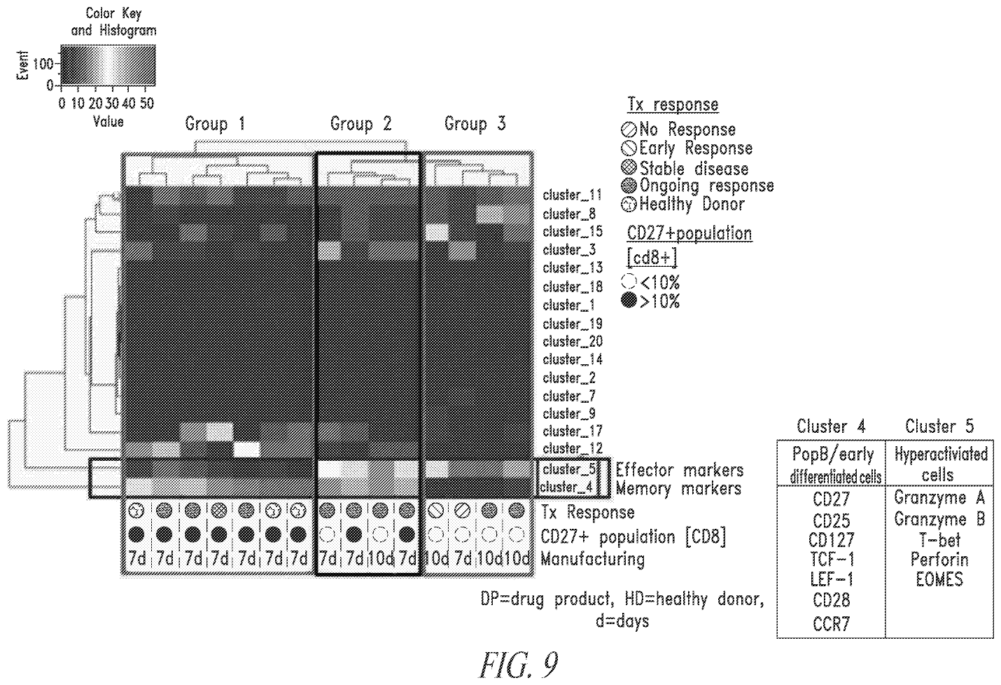

The CD8⁺ T cell data generated in Example 9 was analyzed using FlowSOM. FlowSOM identified 20 distinct T cell clusters. Three major groups of T cells were identified based on clusters 4 (enriched in memory T cell markers, e.g., CD27, CD25, CD127, TCF1, LEF1, CD28, CCR7) and cluster 5 (enriched in effector T cell markers e.g., Granzyme A, Granzyme B, Perforin, T-bet, EOMES). %CD27⁺CD8⁺ anti-BCMA CAR T cells, manufacturing method, and clinical responses for subjects treated with the anti-BCMA CAR T cells were analyzed. The 7 day manufacturing process generally resulted in anti-BCMA CAR T cells with increased expression of T cell memory markers and increased population of CD27⁺ enriched cells compared to the 10 day manufacturing process. FIG. 9.

### Example 11

Anti-BCMA CAR T Cell Gene Expression Analysis

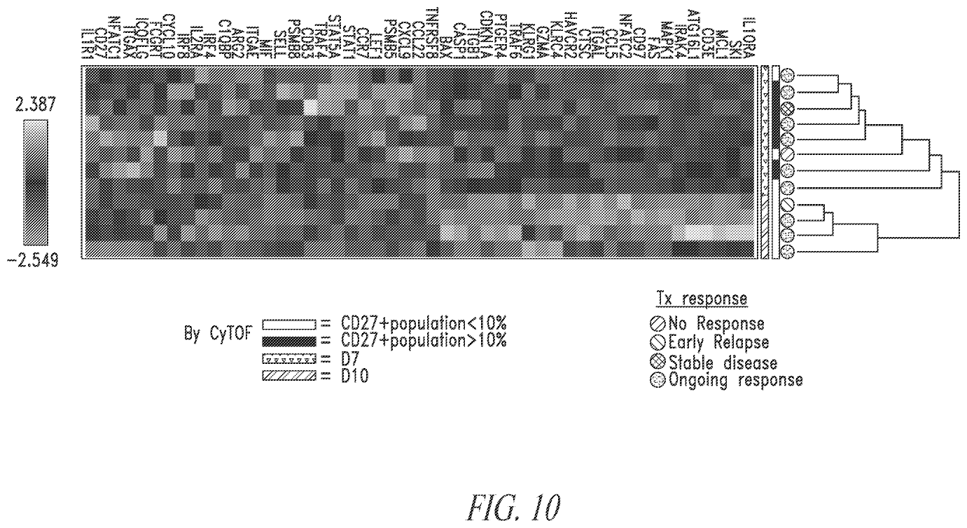

Twelve multiple myeloma donor PBMC cell lots were used to manufacture anti-BMCA CAR T cells using a 7 day (n=8) or 10 day (n=4) manufacturing process described in Example 1 in the presence of the PI3K inhibitor ZSTK474. About 100 ng of total RNA was extracted from anti-BCMA CAR T cell DPs and mixed with the Immunology V2 probe kit from Nanostring. The data were QC′d in NSolver software (Nanostring) and differential gene expression analysis was performed. A heatmap of the top 25 differentially expressed genes (p-value 0.05) between the 7 day and 10 day manufacturing processes was generated. %CD27⁺ anti-BCMA CAR T cells, manufacturing method, and clinical responses for subjects treated with the anti-BCMA CAR T cells were analyzed. The 7 day manufacturing process generally resulted in anti-BCMA CAR T cells with increased expression of T cell memory markers and increased population of CD27⁺ enriched cells compared to the 10 day manufacturing process. FIG. 10.

### Example 12

### Anti-BCMA CAR T Cell Gene Expression Analysis

Five multiple myeloma donor PBMC cell lots were each split into two groups, one group was used to manufacture anti-BMCA CAR T cells using a 7 day manufacturing process and the other group was used to manufacture anti-BMCA CAR T cells using a 10 day manufacturing process. CAR T cells were manufactured in the presence of the PI3K inhibitor ZSTK4 as described in Example 1.

About 100 ng of total RNA was extracted from anti-BCMA CAR T cell DPs and mixed with the Immunology V2 probe kit from Nanostring. The data were QC'd in NSolver software (Nanostring) and differential gene expression analysis was performed.

RNA sequencing (RNA-Seq) was also performed using aliquots of anti-BCMA CAR T cell DP total RNA. Cells were thawed/washed/counted and tested for viability (>70% viability required). Total RNA from 2-3×10⁶ cells was extracted using TRIAZOL. RNA was harvested using phenol/chloroform extraction and Qiagen miRNA-easy kit for total RNA. RNA was isolated using a poly-A bead capture strategy. RNA quality/quantity was determined by the Tapestation 2200 (RIN values >7 required). Sequencing libraries were prepared by Illumina TruSeq RNA. Libraries were quality checked by Tapestation 2200 (DNA kit) and sequenced using a NextSeq550 instrument. Data were analyzed using QC/Alignment methods.

The top 11 upregulated genes and the top 9 down regulated genes, by fold change (FC), relative to the day 7 manufacturing process is shown in Table 1.

| | FC Increase: | | FC Increase: |
|---|---|---|---|
| Gene | Day 7/Day 10 | Gene | Day 7/Day 10 |
| NR4A2 | 2.6 | NQO1 | 2.0 |
| LY9 | 2.5 | CCNA1 | 1.9 |
| LIN7A | 2.5 | IL17F | 1.9 |
| WNT5B | 2.3 | EMP1 | 1.9 |
| BCL6 | 2.3 | SNHG19 | 1.9 |
| EGR1 | 2.3 | PRR22 | 1.9 |
| EGR2 | 2.1 | ILDR2 | 1.7 |
| ATF3 | 2.1 | ATAD3 | 1.7 |
| CCL1 | 2.1 | NKD2 | 1.7 |
| IL-1A | 1.9 | WDR62 | 1.7 |
| CCL5 | 1.7 | | |

### Example 13

### Anti-BCMA CAR T Cell Gene Expression Analysis

Five multiple myeloma donor PBMC cell lots were each split into two groups, one group was used to manufacture anti-BMCA CAR T cells using a 7 day manufacturing process and the other group was used to manufacture anti-BMCA CAR T cells using a 10 day manufacturing process. CAR T cells were manufactured in the presence of the PI3K inhibitor ZSTK4 as described in Example 1.

RNA sequencing (RNA-Seq) was performed using aliquots of anti-BCMA CAR T cell DP total RNA. Cells were thawed/washed/counted and tested for viability (>70% viability required). Total RNA from 2-3×10⁶ cells was extracted using TRIAZOL. RNA was harvested using phenol/chloroform extraction and Qiagen miRNA-easy kit for total RNA. RiboErase was used for rRNA depletion. RNA quality/quantity is determined by the Tapestation 2200 (RN values >7 required). RNA quality/quantity was determined by the Tapestation 2200 (RN values >7 required). Sequencing libraries were prepared by Illumina TruSeq RNA. Libraries were quality checked by Tapestation 2200 (DNA kit) and sequenced using a NextSeq550 instrument. Data were analyzed using QC/Alignment methods.

CCL1, NR4A2, ATF3, CCL5, and WNT5B were among the top 25 upregulated genes and NKD2 and NQO1 were among the top 10 down regulated genes, by fold change (FC), relative to the day 7 manufacturing process.

### Example 14

Anti-BCMA Cart Cell Therapy

PBMCs from multiple myeloma patients were harvested, washed and resuspended in T cell growth medium (TCGM) with 250IU IU/mL IL-2. Pre- and post-wash cell counts, viability, and PBMC flow cytometry analyses were performed. Washed PBMCs were cryopreserved until activation or used fresh. On day 0, T cells were activated and stimulated by culturing the PBMCs in TCGM with 250 IU/mL IL-2, 50 ng/mL of anti-CD3 antibody, and 50 ng/mL of anti-CD28 antibody and cultured for about 18-24 hours. The PBMC culture was transduced with a lentivirus encoding an anti-BCMA CAR (e.g., SEQ ID NO: 1, SEQ ID NO: 2) for about 18 to about 24 hours. The PBMC culture was then cultured for T cell expansion in TCGM containing 250 IU/mL of IL-2 for 9 days (10 day manufacturing process). Expanded cells were recovered, washed and cryopreserved in a controlled rate freezer at a temperature of at least -80° C. and subsequently stored in the vapor phase of a liquid nitrogen storage tank.

The frozen cells were subsequently thawed/washed/counted and tested for viability (>70% viability required). Cells were then either used for CyTOF experiments or frozen down as cell pellets conserved in TRIzol for later RNA extraction and gene expression analysis.

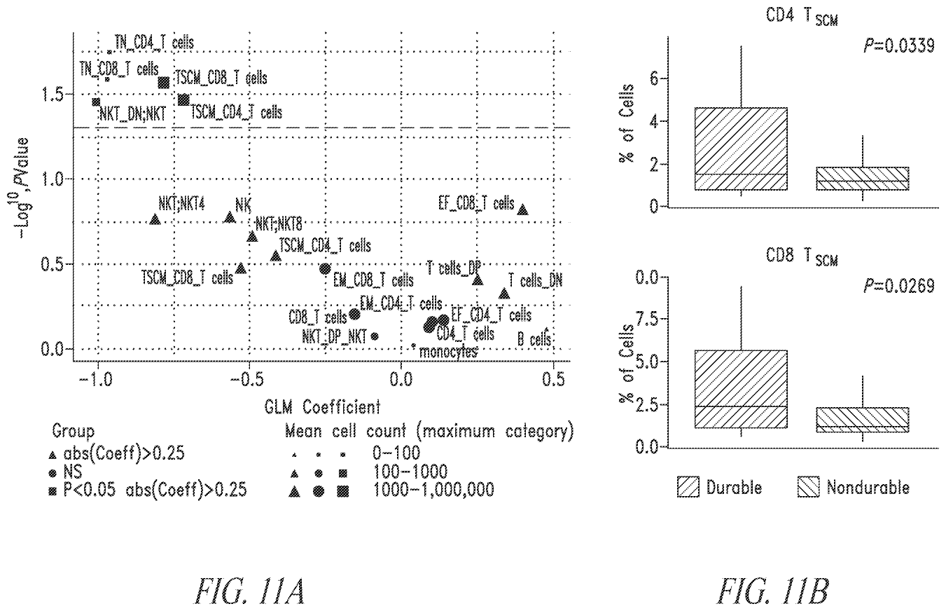

EXPT. 1. Cells were stained with metal labeled anti-human antibodies against T cell markers and analyzed by using a Fluidigm CyTOF Helios Mass Cytometer. Protein marker expression was gated on a single marker basis compared to established negative populations in a reference sample that was spiked into each sample prior to antibody-staining. Cells were classified into memory cell types using a combination of markers and gated on positive marker expression by the silhouette method. Memory populations for CD4 and CD8 T cells, respectively were gated by using following marker combinations: TNaive (CCR7+CD45RO−CD95−), T_{SCM} (CCR7+CD45RO−CD95+), T_{CM} (CCR7+CD45RO+CD95+), TEM (CCR7−CD45RO+CD95+), TEF (CCR7-CD45RO-CD95+). Major immune populations were gated by using following marker combinations: CD4 T cells (CD3+CD4+CDS-CD14−CD19−CD56−), CD8 T cells (CD3+CD4−CD8+CD14−CD19−CD56−), NK cells (CD3−CD19−CD14−CD56+), NKT cells (CD3+CD56+CD19−CD14−), B cells (CD3−CD19+CD14−CD56−) and Monocytes (CD3−CD19−CD14+CD56−). Differential abundance of cell proportions was inferred using a quasi-binomial generalized linear model adjusted for sex. Difference in proportions for individual markers in each cell type was inferred using a Wilcoxon rank sum test. CAR T cell compositions were compared between patients with a duration of response superior to 18 months (durable responders) compared to all patients who had a duration of response of less than 18 months (nondurable responders). FIGS. 11A and 11B.

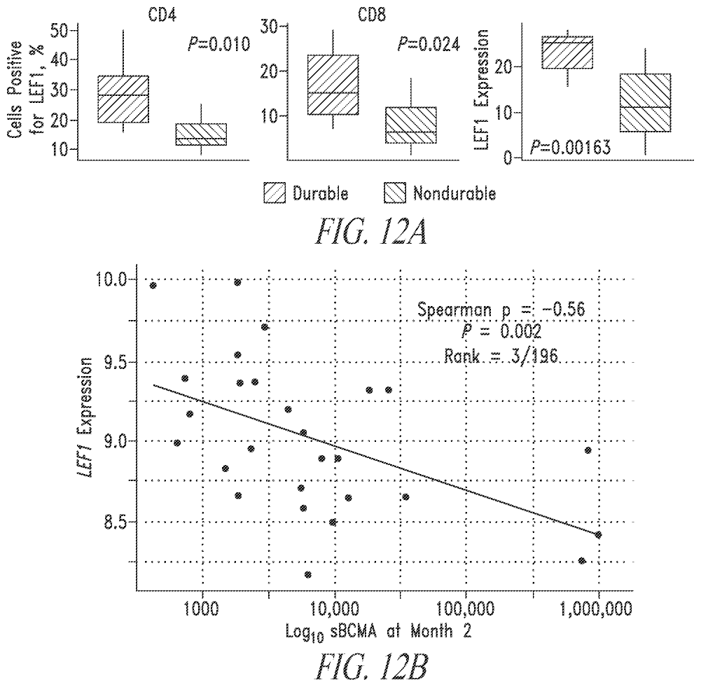

EXPT 2. Cells were stained with metal labeled anti-human antibodies against T cell markers including LEF-1 and analyzed by CyTOF. CyTOF data were quality checked and analyzed to result in expression of individual markers for CD4 and CD8 immune cell populations. Difference in proportions for individual markers in each cell type was inferred using a Wilcoxon rank sum test. Analysis of gene-level counts from drug product samples was performed using differential expression analysis in durable compared to nondurable responders and male versus female sex. FIG. 12A.

RNA was harvested using phenol/chloroform extraction and Qiagen miRNA-easy kit for total RNA and rRNA was depleted using the Kapa RNA HyperPrep Kits with RiboErase. RNA quality/quantity was determined by the Tapestation 2200 (RNA Integrity Number, or RIN, >7 required). Sequencing libraries were prepared using an Illumina TruSeq RNA Library Preparation Kit. Library quality and quantity were determined by Tapestation 2200 (DNA kit) and sequenced using an Illumina NextSeq550 instrument. Sequencing data were analyzed. The correlation of LEFT gene expression with serum BCMA (sBCMA) levels was determined using Spearman rank correlation. FIG. 12B.

### Example 15

Anti-BCMA Cart Cell Therapy

PBMCs from multiple myeloma patients were harvested, washed and resuspended in T cell growth medium (TCGM) with 250IU IU/mL IL-2. Pre- and post-wash cell counts, viability, and PBMC flow cytometry analyses were performed. Washed PBMCs were cryopreserved until activation or used fresh. On day 0, T cells were activated and stimulated by culturing the PBMCs in TCGM with 250 IU/mL IL-2, 50 ng/mL of anti-CD3 antibody, 50 ng/mL of anti-CD28 antibody and cultured for about 18-24 hours in the presence of 1 μM ZSTK474 (PI3K inhibitor, CAS NO. 475110-96-4). The PBMC culture was transduced with a lentivirus encoding an anti-BCMA CAR (e.g., SEQ ID NO: 1, SEQ ID NO: 2) for about 18 to about 24 hours. The PBMC culture was then cultured for T cell expansion in TCGM containing 250 IU/mL of IL-2 and 1 μM ZSTK474 for 9 days (10 day manufacturing processes). Expanded cells were recovered and washed and cryopreserved in a controlled rate freezer at a temperature of at least −80° C. and subsequently stored in the vapor phase of a liquid nitrogen storage tank.

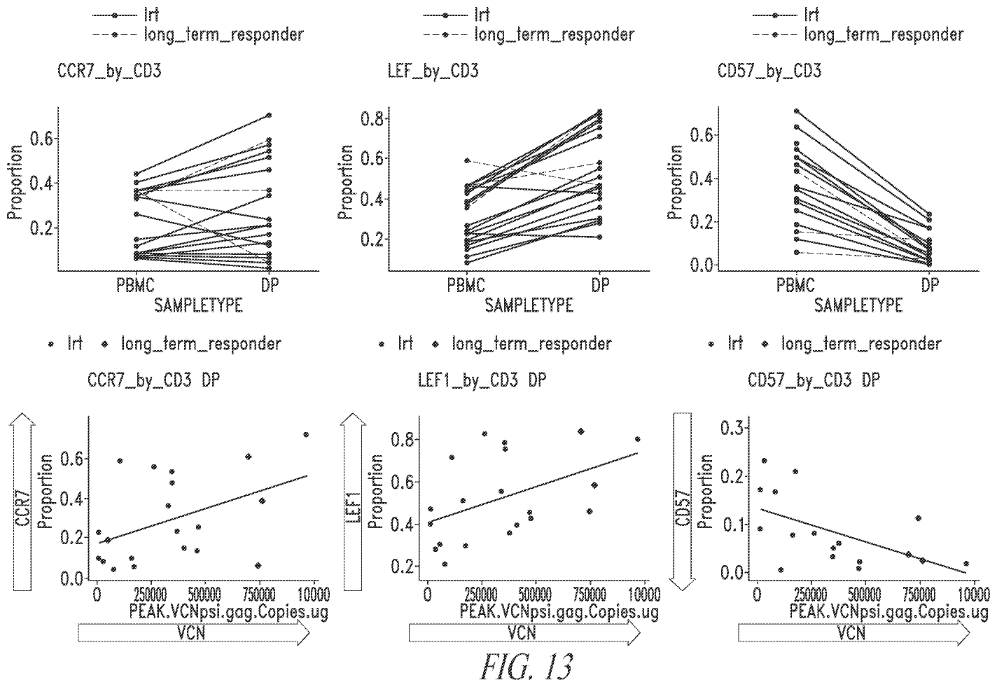

Cryopreserved samples were thawed and stained with metal labeled anti-human antibodies against T cell markers, including CD3, CD27, CCR7 and CD57. Labeled cells were analyzed by using a Fluidigm CyTOF Helios Mass Cytometer. Manual analysis of CyTOF phenotyping was performed using the FlowJo software package. Expression of protein markers was gated on a single marker basis based on established negative populations in a reference sample that was spiked into each subject sample prior to antibody-staining. The percentage of CD3+ live cells expressing CCR7 (FIG. 13, top left panel), LEFT (FIG. 13, top center panel) and CD57 (FIG. 13, top right panel) is shown between the PBMC and the DP. This demonstrated that the PI3-K inhibitor-based manufacturing process enriches for early memory, less differentiated cells.

The percentage of CD3+ live cells expressing CCR7 (FIG. 13, bottom left panel), LEF-1 (FIG. 13, bottom center panel) and CD57 (FIG. 13, bottom right panel) is shown on the y axis. The maximum vector copy number (VCN) determined by PCR on CD3+ cells extracted from whole blood at various time points after infusion, is shown on the x axis. These graphs show a positive correlation in the maximal expansion of the anti-BCMA CAR+cells post-infusion and percentage of CD3+ DP cells expressing LEF-1, as well as a negative correlation with the percentage of CD3+ DP expressing CD57. This indicates an enrichment of CCR7 and LEF-2 in the DP leads to a more robust expansion of the anti-BCMA CARs in vivo.

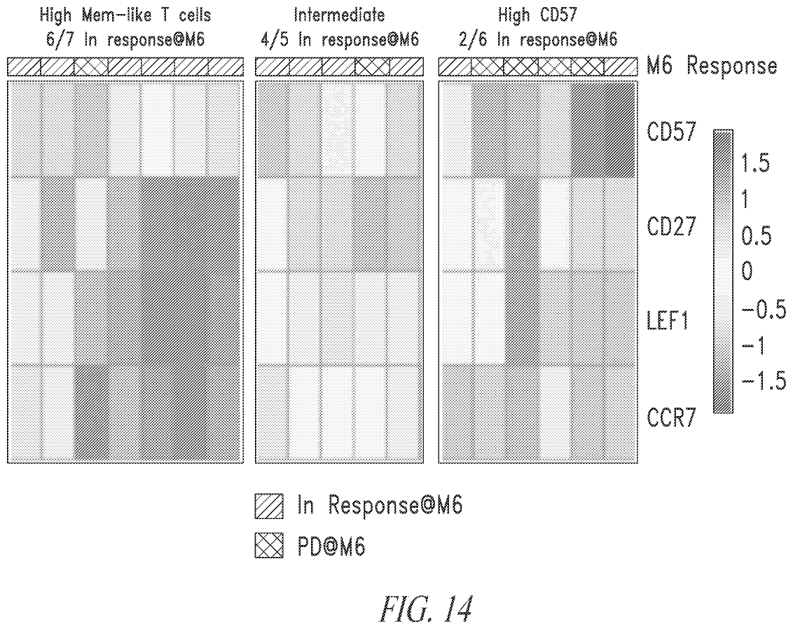

The percentage of CD3+ live cells expressing CD57 (marker of senescence), LEF-1, CCR7 and CD27 (memory cells) are shown as a clustered heatmap. FIG. 14. Red indicates a relatively higher proportion of cells in the sample compared to other samples for the marker. Blue indicates a relatively lower proportion of cells in the sample compared to other samples for the marker. The data were grouped using average linkage hierarchical clustering and the top 3 clusters as determined by the cluster dendrograms were associated with patients' clinical response at 6 months (progressive disease or not). Only patients with available follow-up data to make a clinical evaluation of response at 6 months were included in this analysis. The unsupervised clustering shows association of high CD57 expressing, low LEF-1/CCR7/CD27 expressing group with progressors at 6 months (4/6 progressing), whereas the group with high LEF-1/CCR7/CD27 expression and low CD57 expression is predominantly non-progressors (1/7 progressing). The intermediate group has 1/5 progressors. This demonstrates the correlative relationship between memory and senescent markers in drug products and sustained clinical response.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A cGMP manufactured population of anti-B cell maturation antigen (BCMA) chimeric antigen receptor (CAR) T cells that comprises at least 10% CD27⁺ anti-BCMA CAR T cells.

2. The cGMP manufactured population of anti-BCMA CAR T cells of claim 1, wherein the population comprises at least 15% CD27⁺ anti-BCMA CAR T cells.

3. The cGMP manufactured population of anti-BCMA CAR T cells of claim 1, wherein the population comprises at least 20% CD27⁺ anti-BCMA CAR T cells.

4. The cGMP manufactured population of anti-BCMA CAR T cells of claim 1, wherein the population comprises at least 25% CD27⁺ anti-BCMA CAR T cells.

5. The cGMP manufactured population of anti-BCMA CAR T cells of claim 1, wherein the population comprises at least 30% CD27⁺ anti-BCMA CAR T cells.

6. The cGMP manufactured population of anti-BCMA CAR T cells of any one of claims 1 to 5, wherein the CD27⁺ anti-BCMA CAR T cells are LEF1⁺ and/or CCR7⁺ and/or TCF1⁺ anti-BCMA CAR T cells.

7. The cGMP manufactured population of anti-BCMA CAR T cells of any one of claims 1 to 5, wherein the CD27⁺ anti-BCMA CAR T cells are LEF1⁺ and CCR7⁺ and TCF1⁺ anti-BCMA CAR T cells.

8. The cGMP manufactured population of anti-BCMA CAR T cells of any one of claims 1 to 5, wherein the CD27⁺ anti-BCMA CAR T cells comprise CD4⁺ anti-BCMA CAR T cells.

9. The cGMP manufactured population of anti-BCMA CAR T cells of any one of claims 1 to 5, wherein the CD27⁺ anti-BCMA CAR T cells comprise CD8⁺ anti-BCMA CAR T cells.

10. The cGMP manufactured population of anti-BCMA CAR T cells of any one of claims 1 to 5, wherein the CD27⁺ anti-BCMA CAR T cells comprise CD4⁺ and CD8⁺ anti-BCMA CAR T cells.

11. A cGMP manufactured population of anti- BCMA CAR T cells that comprises at least 10% LEF1⁺ and/or CCR7⁺ and TCF1⁺ anti-BCMA CAR T cells.

12. The cGMP manufactured population of anti-BCMA CAR T cells of claim 11, wherein the population comprises at least 15% LEF1⁺ and/or CCR7⁺ and TCF1⁺ anti-BCMA CAR T cells.

13. The cGMP manufactured population of anti-BCMA CAR T cells of claim 11, wherein the population comprises at least 20% LEF1⁺ and/or CCR7⁺ and TCF1⁺ anti-BCMA CAR T cells.

14. The cGMP manufactured population of anti-BCMA CAR T cells of claim 11, wherein the population comprises at least 25% LEF1⁺ and/or CCR7⁺ and TCF1⁺ anti-BCMA CAR T cells.

15. The cGMP manufactured population of anti-BCMA CAR T cells of claim 11, wherein the population comprises at least 30% LEF1⁺ and/or CCR7⁺ and TCF1⁺ anti-BCMA CAR T cells.

16. The cGMP manufactured population of anti-BCMA CAR T cells of any one of claims 11 to 15, wherein the LEF1⁺ and/or CCR7⁺ and/or TCF1⁺ anti-BCMA CAR T cells are CD27⁺ anti-BCMA CAR T cells.

17. The cGMP manufactured population of anti-BCMA CAR T cells of any one of claims 11 to 15, wherein the LEF1⁺ and/or CCR7⁺ and/or TCF1⁺ anti-BCMA CAR T cells are LEF1⁺CCR7⁺TCF1⁺CD27⁺ anti-BCMA CAR T cells.

18. The cGMP manufactured population of anti-BCMA CAR T cells of any one of claims 11 to 15, wherein the anti-BCMA CAR T cells comprise CD4⁺ anti-BCMA CAR T cells.

19. The cGMP manufactured population of anti-BCMA CAR T cells of any one of claims 11 to 15, wherein the anti-BCMA CAR T cells comprise CD8⁺ anti-BCMA CAR T cells.

20. The cGMP manufactured population of anti-BCMA CAR T cells of any one of claims 11 to 15, wherein the anti-BCMA CAR T cells comprise CD4⁺ and CD8⁺ anti-BCMA CAR T cells.

21. The cGMP manufactured population of anti-BCMA CAR T cells of any one of claims 1 to 20, wherein the cells were manufactured from a subject that has a multiple myeloma or a lymphoma.

22. The cGMP manufactured population of anti-BCMA CAR T cells of any one of claims 1 to 21, wherein the cells were manufactured from a subject that has relapsed/refractory multiple myeloma.

23. The cGMP manufactured population of anti-BCMA CAR T cells of any one of claims 1 to 22, wherein the cells comprise a lentivirus comprising a polynucleotide encoding the anti-BCMA CAR.

24. The cGMP manufactured population of anti-BCMA CAR T cells of any one of claims 1 to 23, wherein the anti-BCMA CAR comprises the amino acid sequence set forth in SEQ ID NO: 1.

25. The cGMP manufactured population of anti-BCMA CAR T cells of any one of claims 1 to 24, wherein the anti-BCMA CAR is encoded by a polynucleotide sequence set forth in SEQ ID NO: 2.

26. The cGMP manufactured population of anti-BCMA CAR T cells of any one of claims 1 to 25, wherein the cells are autologous. 27, The cGMP manufactured population of anti-BCMA CAR T cells of any one of claims 1 to 26, wherein the cells are cryopreserved.

28. The cGMP manufactured population of anti-BCMA CAR T cells of any one of claims 1 to 27, wherein the cells are formulated for administration to a subject that has multiple myeloma or lymphoma.

29. Human anti-B cell maturation antigen (BCMA) chimeric antigen receptor (CAR) T cells that have been contacted ex vivo with a phosphatidyl-inositol-3 kinase (PI3K) inhibitor for about 5 to about 7 days, wherein the gene expression of 1, 2, 3, 4, 5, 6, 7, 8, 9, 10, or all of (i) NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B is at least 1.5-fold or at least 2-fold greater in the anti-BCMA CAR T cells than in an anti-BCMA CAR T cells contacted ex vivo with the PI3K inhibitor for about 10 days.

30. Human anti-B cell maturation antigen (BCMA) chimeric antigen receptor (CAR) T cells that have been contacted ex vivo with a phosphatidyl-inositol-3 kinase (PI3K) inhibitor for about 5 to about 7 days, wherein the gene expression of 1, 2, 3, 4, 5, 6, 7, 8, 9, or all of (i)NQ01, CCNA1, IL17F, EMP1, SNHG19, PRR 22, ILDR2, ATAD3, NKD2 and WDR62 or (ii) NKD2 and NQO1 is at least 1.5-fold or at least 2-fold less in the anti-BCMA CAR T cells than in an anti-BCMA CAR T cells contacted ex vivo with the PI3K inhibitor for about 10 days.

31. Human anti-B cell maturation antigen (BCMA) chimeric antigen receptor (CAR) T cells that have been contacted ex vivo with a phosphatidyl-inositol-3 kinase (PI3K) inhibitor for about 5 to about 7 days; wherein the gene expression of each of 1, 2, 3, 4, 5, 6, 7, 8, 9, 10, or all of (i) NR4A2, LY9, LIN7A, WNTSB, BCL6, EGR1, EGR2, ATF3, CCL1. IL-IA, and CCL5 or (ii) CCL1, NR4A2, ATF3, CCL5, and WNTSB is at least 1.5-fold or at least 2-fold greater and the gene expression 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQO1 is at least 1.5-fold or at least 2-fold less, in the anti-BCMA CAR T cells than in an anti-BCMA CAR T cells contacted ex vivo with the PI3K inhibitor for about 10 days.

32. The human anti-BCMA CAR T cells of any one of claims 29 to 31, wherein CD4⁺ anti-BCMA CAR T cells have a central memory T cell (TCM) like phenotype.

33. The human anti-BCMA CAR T cells of any one of claims 29 to 31, wherein CD8⁺ anti-BCMA CAR T cells have a stem cell memory T cell (TSCM) like phenotype.

34. The human anti-BCMA CAR T cells of any one of claims 29 to 31, wherein CD4⁺ anti-BCMA CAR T cells have a TCM like phenotype and CD8⁺ anti-BCMA CAR T cells have a TSCM like phenotype.

35. The human anti-BCMA CAR T cells of any one of claims 29 to 34, wherein the cells were manufactured from a subject that has a multiple myeloma or a lymphoma.

36. The human anti-BCMA CAR T cells of any one of claims 29 to 35, wherein the cells were manufactured from a subject has relapsed/refractory multiple myeloma.

37. The human anti-BCMA CAR T cells of any one of claims 29 to 36, wherein the cells comprise a lentivirus comprising a polynucleotide encoding the anti-BCMA CAR.

38. The human anti-BCMA CAR T cells of any one of claims 29 to 37, wherein the anti-BCMA CAR comprises the amino acid sequence set forth in SEQ ID NO: 1.

39. The human anti-BCMA CAR T cells of any one of claims 29 to 38, wherein the anti-BCMA CAR is encoded by a polynucleotide sequence set forth in SEQ ID NO: 2.

40. The human anti-BCMA CAR T cells of any one of claims 29 to 39, wherein the cells are autologous.

41. The human anti-BCMA CAR T cells of any one of claims 29 to 40, wherein the cells are cryopreserved.

42. The human anti-BCMA CAR T cells of any one of claims 29 to 41, wherein the cells are formulated for administration to a subject that has multiple myeloma or lymphoma.

43. The human anti-BCMA CAR T cells of any one of claims 29 to 42, wherein the PI3K inhibitor is ZSTK474.

44. A pharmaceutical composition comprising a physiologically acceptable excipient and a therapeutically effective amount of the anti-BCMA CAR T cells of any one of claims 29 to 43.

45. The composition of claim 44, wherein the therapeutically effective amount of the anti-BCMA CAR T cells is at least about 5.0×10⁷ anti-BCMA CAR T cells.

46. The composition of claim 44, wherein the therapeutically effective amount of the anti-BCMA CAR T cells is at least about 15.0×10⁷ anti-BCMA CAR T cells.

47. The composition of claim 44, wherein the therapeutically effective amount is at least about 45.0×10⁷ anti-BCMA CAR T cells.

48. The composition of claim 44, wherein the therapeutically effective amount is at least about 80.0×10⁷ anti-BCMA CAR T cells.

49. The composition of any one of claims 44 to 48, formulated in a solution comprising 50:50 PlasmaLyte A to CryoStor CS10.

50. A method of treating a subject that has multiple myeloma or lymphoma with a composition according to any one of claims 44 to 49.

51. The method of claim 50, wherein the subject has relapsed/refractory multiple myeloma.

52. A method for manufacturing anti-BCMA CAR T cells comprising:
(a) activating a population of T cells and stimulating the population of T cells to proliferate;
(b) transducing the T cells with a lentiviral vector encoding an anti-BCMA CAR that comprises the amino acid sequence set forth in SEQ ID NO: 1;
(c) culturing the transduced T cells to proliferate for a period of about 5 to about 7 days;
wherein steps (a)-(c) are performed in the presence of a PI3K inhibitor, and wherein the gene expression of 1, 2, 3, 4, 5, 6, 7, 8, 9, 10, or all of (i) NR4A2, LY9, LIN7A, WNT5B, BCL6, EGR1_{;} EGR2, ATF3, CCL1, IL-1A, and CCL5 or (ii) CCL1, NR4A2, ATF3, CCL5, and WNT5B is at least 1.5-fold or at least two-fold greater in the cultured T cells of step (c) compared to T cells transduced accordingly step (b) and cultured to proliferate for a period of about 10 days.

53. A method for manufacturing anti-BCMA CAR T cells comprising:
(a) activating a population of T cells and stimulating the population of T cells to proliferate;
(b) transducing the T cells with a lentiviral vector encoding an anti-BCMA CAR that comprises the amino acid sequence set forth in SEQ ID NO: 1;
(c) culturing the transduced T cells to proliferate for a period of about 5 to about 7 days;
wherein steps (a)-(c) are performed in the presence of a PI3K inhibitor, and wherein the gene expression of 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQO1 is at least 1.5-fold or at least two-fold less in the cultured T cells of step (c) compared to T cells transduced accordingly step (b) and cultured to proliferate for a period of about 10 days.

54. A method for manufacturing anti-BCMA CAR T cells comprising:
(a) activating a population of T cells and stimulating the population of T cells to proliferate;
(b) transducing the T cells with a lentiviral vector encoding an anti-BCMA CAR that comprises the amino acid sequence set forth in SEQ ID NO: 1;
(c) culturing the transduced T cells to proliferate for a period of about 5 to about 7 days;
wherein steps (a)-(c) are performed in the presence of PI3K inhibitor, and
wherein the gene expression of 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold or at least two-fold greater and the gene expression of 1, 2, 3, 4, 5, 6, 7, 8, 9, or all of (i) NQO1, CCNA1, IL17F, EMP1, SNHG19, PRR 22, ILDR2, ATAD3, NKD2 and WDR62 or (ii) NKD2 and NQO1 is at least 1.5-fold or at least two-fold less, in the cultured T cells of step (c) compared to T cells transduced accordingly step (b) and cultured to proliferate for a period of about 10 days.

55. A method for manufacturing anti-BCMA CAR T cells comprising:
(a) activating a population of T cells and stimulating the population of T cells to proliferate;
(b) transducing the T cells with a lentiviral vector encoding an anti-BCMA CAR that comprises the amino acid sequence set forth in SEQ ID NO: 1;
(c) culturing the transduced T cells to proliferate for a period of about 5 to about 7 days;
wherein steps (a)-(c) are performed in the presence of a PI3K inhibitor, and wherein the proliferated cells are CD27⁺ and/or LEF1⁺ and/or CCR7⁺ and/or TCF1⁺.

56. The method of any one of claims 52 to 55, wherein the anti-BCMA CAR T cells comprise at least 10% CD27⁺ and/or LEF1⁺ and/or CCR7⁺ and/or TCF1⁺T cells.

57. The method of any one of claims 52 to 55, wherein the anti-BCMA CAR T cells comprise at least 15% CD27⁺ and/or LEF1⁺ and/or CCR7⁺ and/or TCF1⁺ cells.

58. The method of any one of claims 52 to 55, wherein the anti-BCMA CAR T cells comprise at least 20% CD27⁺ and/or LEF1⁺ and/or CCR7⁺ and/or TCF1⁺ cells.

59. The method of any one of claims 52 to 55, wherein the anti-BCMA CAR T cells comprise at least 25% CD27⁺ and/or LEF1⁺ and/or CCR7⁺ and/or TCF1⁺ cells.

60. The method of any one of claims 52 to 55, wherein the anti-BCMA CAR T cells comprise at least 30% CD27⁺ and/or LEF1⁺ and/or CCR7⁺ and/or TCF1⁺ cells.

61. The method of any one of claims 52 to 60, wherein the CD27⁺ cells are LEF1⁺ and/or CCR7⁺ and/or TCF1⁺.

62. The method of any one of claims 52 to 60, wherein the CD27⁺ cells are LEF1⁺ and/or CCR7⁺ and TCF1⁺.

63. The method of any one of claims 52 to 62, wherein the CD27⁺ anti-BCMA CAR T cells comprise CD4⁺ anti-BCMA CAR T cells.

64. The method of any one of claims 52 to 62, wherein the CD27⁺ anti-BCMA CAR T cells comprise CD8⁺ anti-BCMA CAR T cells.

65. The method of any one of claims 52 to 62, wherein the CD27⁺ anti-BCMA CAR T cells comprise CD4⁺ and CD8⁺ anti-BCMA CAR T cells.

66. The method of any one of claims 52 to 65, wherein the T cells are autologous.

67. The method of any one of claims 52 to 66, wherein the method further comprises isolating peripheral blood mononuclear cells (PBMCs) as the source of T cells.

68. The method of claim 67, wherein the PBMCs are isolated from a subject that has a multiple myeloma or a lymphoma.

69. The method of claim 68, wherein the subject has relapsed/refractory multiple myeloma.

70. The method of any one of claims 52 to 69, wherein the method further comprises cryopreserving the PBMCs before step (a).

71. The method of any one of claims 52 to 70, wherein the T cells are cryopreserved after step (c).

72. The method of any one of claims 52 to 71, wherein the T cell are activated and simulated to proliferate for about 18 to about 24 hours.

73. The method of any one of claims 52 to 72, wherein activation of the T cells comprises contacting the T cells with an anti-CD3 antibody or antigen binding fragment thereof.

74. The method of claim 73, wherein the anti-CD3 antibody or antigen binding fragment thereof is soluble.

75. The method of claim 73, wherein the anti-CD3 antibody or antigen binding fragment thereof is bound to a surface.

76. The method of claim 75, wherein the surface is a bead, optionally a paramagnetic bead.

77. The method of any one of claims 52 to 76, wherein stimulation of the T cells comprises contacting the T cells with an anti-CD28 antibody or antigen binding fragment thereof.

78. The method of claim 77, wherein the anti-CD28 antibody or antigen binding fragment thereof is soluble.

79. The method of claim 77, wherein the anti-CD28 antibody or antigen binding fragment thereof is bound to a surface.

80. The method of claim 79, wherein the surface is a bead, optionally a paramagnetic bead, optionally the paramagnetic bead bound to the anti-CD3 antibody or antigen binding fragment thereof.

81. The method of any one of claims 52 to 80, wherein the cells are transduced with an HIV-1 derived lentiviral vector.

82. The method of any one of claims 52 to 81, wherein the anti-BCMA CAR is encoded by the polynucleotide sequence set forth in SEQ ID NO: 2.

83. The method of any one of claims 52 to 82, wherein the PI3K inhibitor is ZSTK474.

84. A method for increasing CD4⁺ TCM like anti-BCMA CAR T cells and CD8⁺ TSCM like anti-BCMA CAR T cells in an adoptive cell therapy comprising contacting anti-BCMA CAR T cells ex vivo with a PI3K inhibitor for about 5 to about 7 days, wherein the number of CD4⁺ TCM like anti-BCMA CAR T cells and CD8⁺ TSCM like anti-BCMA CAR T cells is at least two-fold greater in the anti-BCMA CAR T cells than in anti-BCMA CAR T cells contacted ex vivo with the PI3K inhibitor for about 10 days.

85. The method of claim 84, wherein the anti-BCMA CAR T cells comprise at least 10% CD27⁺ and/or LEF1⁺ and/or CCR7⁺ and/or TCF1⁺ cells.

86. The method of claim 84 or claim 85, wherein the anti-BCMA CAR T cells comprise at least 15% CD27⁺ and/or LEF1⁺ and/or CCR7⁺ and/or TCF1⁺ cells.

87. The method of any one of claims 84 to 86, wherein the anti-BCMA CAR T cells comprise at least 20% CD27⁺ and/or LEF1⁺ and/or CCR7⁺ and/or TCF1⁺ cells.

88. The method of any one of claims 84 to 87, wherein the anti-BCMA CAR T cells comprise at least 25% CD27⁺ and/or LEF1⁺ and/or CCR7⁺ and/or TCF1⁺T cells.

89. The method of any one of claims 84 to 88, wherein the anti-BCMA CAR T cells comprise at least 30% CD27⁺ and/or LEF1⁺ and/or CCR7⁺ and/or TCF1⁺T cells.

90. The method of any one of claims 84 to 89, wherein the T cells are autologous.

91. The method of any one of claims 84 to 90, wherein the method further comprises isolating peripheral blood mononuclear cells (PBMCs) as the source of T cells.

92. The method of claim 91, wherein the PBMCs are isolated from a subject that has a multiple myeloma or a lymphoma.

93. The method of claim 92, wherein the subject has relapsed/refractory multiple myeloma.

94. The method of any one of claims 84 to 93, wherein the anti-BCMA CAR T cells comprise an HIV-1 derived lentiviral vector.

95. The method of any one of claims 84 to 94, wherein the anti-BCMA CAR comprises the amino acid sequence set forth in SEQ ID NO: 1.

96. The method of any one of claims 84 to 95, wherein the anti-BCMA CAR is encoded by the polynucleotide sequence set forth in SEQ ID NO: 2.

97. A pharmaceutical composition comprising a pharmaceutically acceptable excipient and a therapeutically effective amount of the anti-BCMA CAR T cells according to the methods of any one of claims 52 to 83.

98. A pharmaceutical composition comprising a pharmaceutically acceptable excipient and a therapeutically effective amount of the CD4⁺ TCM anti-BCMA CAR T cells and CD8⁺ TSCM anti-BCMA CAR T cells according to any one of claims 84 to 96.

99. A method of treating a subject that has multiple myeloma or lymphoma with a composition according to claim 97 or claim 98.

100. The method of claim 99, wherein the subject has relapsed/refractory multiple myeloma.

101. A method for increasing the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB in anti-BCMA CAR T cells comprising contacting anti-BCMA CAR T cells ex vivo with a PI3K inhibitor for about 5 to about 7 days, wherein the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater in the anti-BCMA CAR T cells than in anti-BCMA CAR T cells contacted ex vivo with the PI3K inhibitor for about 10 days.

102. A method for decreasing the gene expression of each of (i) NQO1, CCNA1, IL17F, EMP1, SNHG19, PRR 22, ILDR2, ATAD3, NKD2 and WDR62 or (ii) NKD2 and NQO1 in anti-BCMA CAR T cells comprising contacting anti-BCMA CAR T cells ex vivo with a PI3K inhibitor for about 5 to about 7 days, wherein the gene expression of each of (i) NQO1, CCNA1, IL17F, EMP1, SNHG19, PRR 22, ILDR2, ATAD3, NKD2 and WDR62 or (ii) NKD2 and NQO1 is at least 1.5-fold less in the anti-BCMA CAR T cells than in anti-BCMA CAR T cells contacted ex vivo with the PI3K inhibitor for about 10 days.

103. A method for increasing the gene expression of each of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB and decreasing the gene expression of each of (i) NQO1, CCNA1, IL17F, EMP1, SNHG19, PRR 22, ILDR2, ATAD3, NKD2 and WDR62 or (ii) NKD2 and NQO1 in anti-BCMA CAR T cells comprising contacting anti-BCMA CAR T cells ex vivo with a PI3K inhibitor for about 5 to about 7 days, wherein the gene expression of each of (i)NR4A2, LY9, LIN7A, WNT5B, BCL6, EGR1, EGR2, ATF3, CCL1, IL-1A, and CCL5 or (ii) CCL1, NR4A2, ATF3, CCL5, and WNT5B is at least 1.5-fold greater and the gene expression of each of (i) NQO1, CCNA1, IL17F, EMP1, SNHG19, PRR 22, ILDR2, ATAD3, NKD2 and WDR62 or (ii) NKD2 and NQO1 is at least 1.5-fold less, in the anti-BCMA CAR T cells than in anti-BCMA CAR T cells contacted ex vivo with the PI3K inhibitor for about 10 days.

104. A method for increasing the therapeutic efficacy of anti-BCMA CAR T cells comprising contacting anti-BCMA CAR T cells ex vivo with a PI3K inhibitor for about 5 to about 7 days, wherein the increase in therapeutic efficacy is indicated by an increase in gene expression of each of 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater in the anti-BCMA CAR T cells compared to anti-BCMA CAR T cells contacted ex vivo with the PI3K inhibitor for about 10 days.

105. A method for increasing the therapeutic efficacy of anti-BCMA CAR T cells comprising contacting anti-BCMA CAR T cells ex vivo with a PI3K inhibitor for about 5 to about 7 days, wherein the increase in therapeutic efficacy is indicated by a decrease in gene expression of each of (i) NQ01, CCNA1, IL17F, EMP1, SNHG19, PRR 22, ILDR2, ATAD3, NKD2 and WDR62 or (ii) NKD2 and NQ01 is at least 1.5-fold less in the anti-BCMA CAR T cells compared to anti-BCMA CAR T cells contacted ex vivo with the PI3K inhibitor for about 10 days.

106. A method for increasing the therapeutic efficacy of anti-BCMA CAR T cells comprising contacting anti-BCMA CAR T cells ex vivo with a PI3K inhibitor for about 5 to about 7 days, wherein the increase in therapeutic efficacy is indicated by an increase in gene expression of each 1, 2, 3, 4, 5, 6, 7, 8, 9, 10, or all of (i) NR4A2, LY9, LIN7A, WNTSB, BCL6, EGR1, EGR2, ATF3, CCL1, IL-1A, and CCL5 or (ii) CCL1, NR4A2, ATF3, CCL5, and WNTSB is at least 1.5-fold greater and a decrease in gene expression of each of (i) NQ01, CCNA1, IL17F, EMP1, SNHG19, PRR 22, ILDR2, ATAD3, NKD2 and WDR62 or (ii) NKD2 and NQ01 is at least 1.5-fold less, in the anti-BCMA CAR T cells compared to anti-BCMA CAR T cells contacted ex vivo with the PI3K inhibitor for about 10 days.

107. The method of any one of claims 101 to 106, wherein the anti-BCMA CAR T cells are from a subject that has a multiple myeloma or a lymphoma.

108. The method of any one of claims 101 to 107, wherein the anti-BCMA CAR T cells are from a subject has relapsed/refractory multiple myeloma.

109. The method of any one of claims 101 to 108, wherein the anti-BCMA CAR T cells comprises an HIV-1 derived lentiviral vector comprising a polynucleotide encoding the anti-BCMA CAR.

110. The method of any one of claims 101 to 109, wherein the anti-BCMA CAR comprises the amino acid sequence set forth in SEQ ID NO: 1.

111. The method of any one of claims 101 to 110, wherein the anti-BCMA CAR is encoded by a polynucleotide sequence set forth in SEQ ID NO: 2.

112. The method of any one of claims 101 to 111, wherein the anti-BCMA CAR T cells are autologous.

113. The method of any one of claims 101 to 112, wherein the PI3K inhibitor is ZSTK474.
